(19) United States
(12) Patent Application Publication
(10) Pub. No.: US 2026/0091039 A1
(43) Pub. Date: Apr. 2, 2026

(54) HYDROCHLORIC ACID SALT OF A 5-HT2C AGONIST

(71) Applicant: Arena Pharmaceuticals, Inc., New York, NY (US)

(72) Inventor: Anthony Clyde Blackburn, San Diego, CA (US)

(21) Appl. No.: 19/344,622

(22) Filed: Sep. 30, 2025

Related U.S. Application Data
(60) Provisional application No. 63/701,650, filed on Oct. 1, 2024.

(51) Int. Cl. A61K 31/5517; C07D 487/04

(52) U.S. Cl. CPC A61K 31/5517; C07D 487/04

(57) ABSTRACT

Provided is crystalline (R)—N-(2,2-difluoroethyl)-7-methyl-1,2,3,4,6,7-hexahydro-[1,4]diazepino[6,7,1-hi]indole-8-carboxamide (Compound 1) hydrochloric acid (HCl) salt. Also are provided pharmaceutical compositions comprising the salt form and methods for its use.

The developmental and epileptic encephalopathies (DEE) are a heterogeneous group of rare neurodevelopmental disorders. They are characterized by early-onset seizures that are often intractable and are associated with electroencephalographic abnormalities, developmental delay or regression that can worsen over time, and in some cases, early death. These disorders are generally diagnosed in childhood and adolescence; they vary in their etiologies, seizure types, electroencephalographic patterns, cognitive deficits, and prognosis. The International League Against Epilepsy recently expanded this definition to include disorders that may result in developmental delay before epilepsy onset and used the term of DEE to encompass this broader population.

5-HT2 receptor agonists have been shown to be efficacious treatments for a variety of motor seizures and seizure disorders. Specifically, low dose fenfluramine (Fintepla®), a mixed 5-HT2C, 5-HT2B, and 5-HT2A receptor agonist, has recently been approved for the treatment of Dravet syndrome (Food and Drug Administration [FDA] approved label June 2020), and it was shown to reduce the frequency of drop seizures in patients with Lennox-Gastaut Syndrome in Phase 3 studies. However, Fintepla® received a Boxed Warning requiring cardiac monitoring because of the association between serotonergic drugs with 5-HT2B receptor agonist activity and valvular heart disease.

The 5-HT_{2C} receptor agonist (R)—N-(2,2-difluoroethyl)-7-methyl-1,2,3,4,6,7-hexahydro-[1,4]diazepino[6,7,1-hi]indole-8-carboxamide (see U.S. Pat. No. 10,392,390) is in clinical trials for the treatment of DEE and related seizure disorders. US Pat. App. Pub. 2023/0293546 sets out TID dosing regimens (including up-titration and down-titration) at dosage amounts of 3, 6, 9 12, 18, and 24 mg and reports PK data including a half-life at steady state ranging from 4.81 to 6.50 hours.

There is a significant unmet need for safe, effective, and tolerable treatment for DEE. There also remains a need for alternative compounds for the treatment of diseases and disorders related to the 5-HT_{2C} receptor. The compounds described herein are 5-HT_{2C} receptor agonists that satisfy this need and provide related advantages as well. The present disclosure satisfies this need and provides related advantages as well.

### SUMMARY

Provided is crystalline (R)—N-(2,2-difluoroethyl)-7-methyl-1,2,3,4,6,7-hexahydro-[1,4]diazepino[6,7,1-hi]indole-8-carboxamide (Compound 1) hydrochloric acid (HCl) salt.

Also provided are pharmaceutical compositions comprising the salt form and methods for its use.

These and other aspects of the invention disclosed herein will be set forth in greater detail as the patent disclosure proceeds.

### BRIEF DESCRIPTION OF THE DRAWINGS

FIG. 1 shows an XRPD pattern for a sample containing Compound 1 mono-HCl salt batch 1. X-axis: diffraction angle (°2θ); Y-axis: intensity (counts).

FIG. 2 shows a DSC thermogram (solid line) and a TGA thermogram (dashed line) for a sample containing Compound 1 mono-HCl salt batch 1. X-axis: Temperature (° C.); Y-axis: normalized heat flow (W/g).

FIG. 3 shows a GVS kinetic plot for a sample containing Compound 1 mono-HCl salt batch 1. Y-axis: Mass (mg), actual RH; X-axis: Time (min).

FIG. 4 shows a ball and stick diagram of Compound 1 mono-HCl salt.

FIG. 5 shows an overlay of a simulated XRPD pattern of Compound 1 mono-HCl salt at low temperature (upper) and an XRPD pattern for a sample containing Compound 1 mono-HCl salt measured at RT (lower). X-axis: diffraction angle (°2θ)

FIG. 6 Shows an XRPD pattern for a sample containing Compound 1 mono-HCl salt batch 2. X-axis: diffraction angle (°2θ); Y-axis: intensity (counts).

FIG. 7 shows a DSC thermogram (solid line) and a TGA thermogram (dashed line) for a sample containing Compound 1 mono-HCl salt batch 2. X-axis: Temperature (° C.); Y-axis: normalized heat flow (W/g).

FIG. 8. Shows a GVS (also referred to as Dynamic Vapor Sorption (DVS)) kinetic plot for a sample containing Compound 1 mono-HCl salt batch 2. X-axis: Time (min); Y-axis (left): Change in mass (%); Y-axis (right): Relative humidity (%).

### DETAILED DESCRIPTION

As used in the present specification, the following words and phrases are generally intended to have the meanings as set forth below, except to the extent that the context in which they are used indicates otherwise.

COMPOUND 1: As used herein, “Compound 1” means (R)—N-(2,2-difluoroethyl)-7-methyl-1,2,3,4,6,7-hexahydro-[1,4]diazepino[6,7,1-hi]indole-8-carboxamide (also known as bexicaserin or LP352).

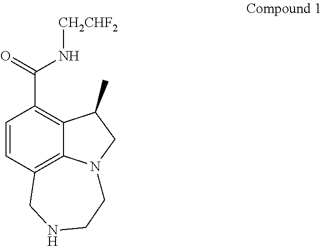

Compound 1 is a potent and selective 5-hydroxytryptamine (HT)_{2C} receptor agonist and exhibits increased selectivity for the ligand binding site of 5-HT_{2C} receptors versus those of 5-HT_{2A} and 5-HT_{2B}. Compound 1 displays a binding affinity of 44 nM at the human 5-HT_{2C} receptor and shows >227-fold selectivity for the 5-HT_{2C} receptor versus 5-HT_{2A} and 5-HT_{2B} in comparison to previously developed agonists such as Fintepla® (low dose fenfluramine).

Methods of use of Compound 1 are disclosed in U.S. Pat. No. 10,392,390, which is incorporated herein by reference in its entirety for all purposes.

CONVULSIVE/MOTOR SEIZURES: As used here, a “convulsive/motor seizure” refers to a tonic-clonic, tonic, tonic-atonic leading to drop, focal motor, epileptic spasms, myoclonic-atonic leading to drop and seizures. Non-convulsive seizures include myoclonic, absence, atypical absence, or atonic seizures and focal seizures without an observable motor component.

CONVULSIVE/MOTOR SEIZURE-FREE DAY: As used herein, a “convulsive/motor seizure-free day” refers to a day for which diary data are available and no convulsive/motor seizures were reported.

DROP SEIZURE: As used herein, the term “drop seizure” refers to a seizure involving the entire body, trunk or head that leads to a fall, injury, slumping in a chair, or head hitting the surface, or could have led to a fall or injury, depending on the position of the subject at the time of the attack or spell.

AGONIST: As used herein, the term “agonist” refers to a moiety that interacts with and activates a receptor, such as the 5-HT_{2C} serotonin receptor, and initiates a physiological or pharmacological response characteristic of that receptor.

ADMINISTERING: As used herein, “administering” means to provide a compound or other therapy, remedy, or treatment such that an individual internalizes a compound.

ORAL or ORALLY: As used herein, “oral” or “orally” refers to administration of a compound or composition to an individual by a route or mode along the alimentary canal. Examples of enteral routes of administration include, without, limitation, oral, as in swallowing solid (e.g., tablet) or liquid (e.g., syrup) forms; sub-lingual (absorption under the tongue); nasojejunal or gastrostomy tubes (into stomach); intraduodenal administration; as well as rectal administration (e.g., suppositories for release and absorption of a compound or composition by in the lower intestinal tract of the alimentary canal).

PRESCRIBING: As used herein, “prescribing” means to order, authorize, or recommend the use of a drug or other therapy, remedy, or treatment. In some embodiments, a health care practitioner can orally advise, recommend, or authorize the use of a compound, dosage regimen or other treatment to an individual. In this case the health care practitioner may or may not provide a prescription for the compound, dosage regimen, or treatment. Further, the health care practitioner may or may not provide the recommended compound or treatment. For example, the health care practitioner can advise the individual where to obtain the compound without providing the compound. In some embodiments, a health care practitioner can provide a prescription for the compound, dosage regimen, or treatment to the individual. For example, a health care practitioner can give a written or oral prescription to an individual. A prescription can be written on paper or on electronic media such as a computer file, for example, on a hand-held computer device. For example, a health care practitioner can transform a piece of paper or electronic media with a prescription for a compound, dosage regimen, or treatment. In addition, a prescription can be called in (oral), faxed in (written), or submitted electronically via the internet to a pharmacy or a dispensary. In some embodiments, a sample of the compound or treatment can be given to the individual. As used herein, giving a sample of a compound constitutes an implicit prescription for the compound. Different health care systems around the world use different methods for prescribing and/or administering compounds or treatments and these methods are encompassed by the disclosure.

A prescription can include, for example, an individual's name and/or identifying information such as date of birth. In addition, for example, a prescription can include: the medication name, medication strength, dose, frequency of administration, route of administration, number or amount to be dispensed, number of refills, physician name, physician signature, and the like. Further, for example, a prescription can include a DEA number and/or state number.

A healthcare practitioner can include, for example, a physician, nurse, nurse practitioner, or other related health care professional who can prescribe or administer compounds (drugs) for the treatment of a condition described herein. In addition, a healthcare practitioner can include anyone who can recommend, prescribe, administer, or prevent an individual from receiving a compound or drug including, for example, an insurance provider.

PREVENT, PREVENTING, OR PREVENTION: As used herein, the term “prevent,” “preventing”, or “prevention,” such as prevention of a particular disorder or the occurrence or onset of one or more symptoms associated with the particular disorder and does not necessarily mean the complete prevention of the disorder. For example, the term “prevent,” “preventing” and “prevention” means the administration of therapy on a prophylactic or preventative basis to an individual who may ultimately manifest at least one symptom of a disease or condition but who has not yet done so. Such individuals can be identified on the basis of risk factors that are known to correlate with the subsequent occurrence of the disease. Alternatively, prevention therapy can be administered without prior identification of a risk factor, as a prophylactic measure. Delaying the onset of at least one symptom can also be considered prevention or prophylaxis.

TREAT, TREATING, OR TREATMENT: As used herein, the term “treat,” “treating”, or “treatment” means the administration of therapy to an individual who already manifests at least one symptom of a disease or condition or who has previously manifested at least one symptom of a disease or condition. For example, “treating” can include alleviating, abating or ameliorating a disease or condition symptoms, preventing additional symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. For example, the term “treating” in reference to a disorder means a reduction in severity of one or more symptoms associated with that particular disorder. Therefore, treating a disorder does not necessarily mean a reduction in severity of all symptoms associated with a disorder and does not necessarily mean a complete reduction in the severity of one or more symptoms associated with a disorder.

TOLERATE: As used herein, an individual is said to “tolerate” a dose of a compound if administration of that dose to that individual does not result in an unacceptable adverse event or an unacceptable combination of adverse events. One of skill in the art will appreciate that tolerance is a subjective measure and that what may be tolerable to one individual may not be tolerable to a different individual. For example, one individual may not be able to tolerate headache, whereas a second individual may find headache tolerable but is not able to tolerate vomiting, whereas for a third individual, either headache alone or vomiting alone is tolerable, but the individual is not able to tolerate the combination of headache and vomiting, even if the severity of each is less than when experienced alone.

INTOLERANCE: As used herein, “intolerance” means significant toxicities and/or tolerability issues that led to a reduction in dose or discontinuation of the medication. “Intolerance” can be replaced herein with the term “unable to tolerate.”

ADVERSE EVENT: As used herein, an “adverse event” is an untoward medical occurrence that is associated with treatment with Compound, 1, including but not limited to, the HCl salt of Compound 1. In one embodiment, an adverse event is selected from: leukopenia, constipation, diarrhea, nausea, abdominal pain, neutropenia, vomiting, back pain, and menstrual disorder. In one embodiment, an adverse event is heart block, for example, a first-degree atrioventricular heart block. In one embodiment, an adverse event is an acute heart rate reduction. In one embodiment, an adverse event is an abnormal pulmonary function test finding, such as an FEV1 below 80%, FVC. In one embodiment, an adverse event is macular edema.

IN NEED OF TREATMENT and IN NEED THEREOF: As used herein, “in need of treatment” and “in need thereof” when referring to treatment are used interchangeably to mean a judgment made by a caregiver (e.g., physician, nurse, nurse practitioner, etc.) that an individual requires or will benefit from treatment. This judgment is made based on a variety of factors that are in the realm of a caregiver's expertise, but that includes the knowledge that the individual is ill, or will become ill, as the result of a disease, condition or disorder that is treatable by the compounds of the invention. Accordingly, the compounds of the invention can be used in a protective or preventive manner; or compounds of the invention can be used to alleviate, inhibit or ameliorate the disease, condition, or disorder.

PATIENT: As used herein, “patient” means any human. In some embodiments, a human individual is referred to a “subject” or “individual.”

DOSE: As used herein, “dose” means a quantity of Compound 1, including but not limited to, the HCl salt of Compound 1, given to the individual for treating or preventing the disease or disorder at one specific time.

DOSAGE: As used herein, “dosage” refers to the amount of Compound 1, including but not limited to, the HCl salt of Compound 1, in one or more doses.

STEADY STATE: As used herein, “steady-state” is reached when the quantity of a drug eliminated in a given unit of time equals the quantity of the drug that reaches systemic circulation in the unit of time. A “steady-state” pharmacokinetic characteristic means the characteristic can be achieved upon reaching steady-state.

THERAPEUTICALLY EFFECTIVE AMOUNT: As used herein, “therapeutically effective amount” of an agent, compound, drug, composition, or combination is an amount which is nontoxic and effective for producing some desired therapeutic effect upon administration to a subject or patient (e.g., a human subject or patient). The precise therapeutically effective amount for a subject may depend upon, e.g., the subject's size and health, the nature and extent of the condition, the therapeutics or combination of therapeutics selected for administration, and other variables known to those of skill in the art. The effective amount for a given situation is determined by routine experimentation and is within the judgment of the clinician. In some embodiments, the therapeutically effective amount is the standard dose.

PHARMACEUTICAL COMPOSITION: As used herein, “pharmaceutical composition” means a composition comprising at least one active ingredient, such as Compound 1, including but not limited to, the HCl salt of Compound 1, whereby the composition is amenable to investigation for a specified, efficacious outcome. Those of ordinary skill in the art will understand and appreciate the techniques appropriate for determining whether an active ingredient has a desired efficacious outcome based upon the needs of the artisan. In some embodiments, the pharmaceutical composition further comprises at least one pharmaceutically acceptable excipient.

When an integer is used in a method disclosed herein, the term “about” can be inserted before the integer.

Throughout this specification, unless the context requires otherwise, the word “comprise”, or variations such as “comprises” or “comprising” will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps, or group of compositions of matter shall be taken to encompass one and a plurality (i.e., one or more) of those steps, compositions of matter, groups of steps, or groups of compositions of matter.

Each embodiment described herein is to be applied mutatis mutandis to each and every other embodiment unless specifically stated otherwise.

Those skilled in the art will appreciate that the invention(s) described herein is susceptible to variations and modifications other than those specifically described. It is to be understood that the invention(s) includes all such variations and modifications. The invention(s) also includes all the steps, features, compositions, and compounds referred to or indicated in this specification, individually or collectively, and any and all combinations or any two or more of said steps or features unless specifically stated otherwise.

The present invention(s) is not to be limited in scope by the specific embodiments described herein, which are intended for the purpose of exemplification only. Functionally equivalent products, compositions, and methods are clearly within the scope of the invention(s), as described herein.

It is appreciated that certain features of the invention(s), which are, for clarity, described in the context of separate embodiments, can also be provided in combination in a single embodiment. Conversely, various features of the invention(s), which are, for brevity, described in the context of a single embodiment, can also be provided separately or in any suitable subcombination. For example, a method that recites prescribing and/or administering can include one method reciting prescribing and the other method reciting administering.

### Compound 1 HCl Salt

Provided is crystalline (R)—N-(2,2-difluoroethyl)-7-methyl-1,2,3,4,6,7-hexahydro-[1,4]diazepino[6,7,1-hi]indole-8-carboxamide (Compound 1) hydrochloric acid (HCl) salt. In some embodiments, the crystalline HCl salt form of Compound 1 is a mono-HCl salt form. In some embodiments, the crystalline mono-HCl salt form of Compound 1 is anhydrous.

The crystalline mono-HCl salt of Compound 1 may be obtained as the direct product of compound synthesis. In the alternative, the free base Compound 1 may be dissolved in a suitable solvent containing HCl (e.g., about 1 molar eq. or slight excess) and the salt isolated by evaporating, cooling, or other means for separating the salt and solvent. In another method, the crystalline mono-HCl salt of Compound 1 may be obtained by starting with a different salt, like the trifluoroacetic acid (TFA) salt (see, U.S. Pat. No. 10,392,390), and replacing the starting acid addition salt with the HCl salt. For example, the TFA salt can be treated with base in a solvent system to generate the free base Compound 1, which may or may not be isolated, and then the free base Compound 1 is treated with HCl (e.g., about 1 molar eq. or slight excess) to form the mono-HCl salt of Compound 1, which can be precipitated from the solution in which it was formed as a crystalline solid.

Crystalline forms described herein can be identified by their unique solid state signature with respect to, for example X-ray powder diffraction (XRPD), and other solid state methods.

For XRPD, the relative intensities of the peaks can vary, depending upon the sample preparation technique, the sample mounting procedure and the particular instrument employed. Moreover, instrument variation and other factors can often affect the 2-theta values. Therefore, the peak assignments of diffraction patterns can vary by plus or minus 0.2 °2-theta (i.e., ±0.2).

As used herein, the term “substantially crystalline,” means a majority of the weight of a sample or preparation of Compound 1 HCl salt is crystalline and the remainder of the sample is a non-crystalline form (e.g., amorphous form) of the same compound. In some embodiments, a substantially crystalline sample has at least about 95% crystallinity (e.g., about 5% of the non-crystalline form of the same compound), at least about 96% crystallinity (e.g., about 4% of the non-crystalline form of the same compound), at least about 97% crystallinity (e.g., about 3% of the non-crystalline form of the same compound), at least about 98% crystallinity (e.g., about 2% of the non-crystalline form of the same compound), at least about 99% crystallinity (e.g., about 1% of the non-crystalline form of the same compound), or about 100% crystallinity (e.g., about 0% of the non-crystalline form of the same compound). In some embodiments, the substantially crystalline form is a single polymorphic form. In some embodiments, the substantially crystalline form includes two or more polymorphic forms.

### Crystalline Compound 1 Mono-HCl Salt

Provided is crystalline Compound 1 mono-HCl salt.

In some embodiments, crystalline Compound 1 mono-HCl salt has an XRPD pattern comprising a peak, in terms of 2-theta, at 10.0°±0.2°.

In some embodiments, crystalline Compound 1 mono-HCl salt has an XRPD pattern comprising peaks, in terms of 2-theta, at 10.0°±0.2°, 14.5°±0.2°, and 22.9°±0.2°.

In some embodiments, crystalline Compound 1 mono-HCl salt has an XRPD pattern comprising a peak, in terms of 2-theta, selected from 10.0°±0.2°, 14.5°±0.2°, 19.9°±0.2°, 22.9°±0.2°, and 24.3°±0.2°.

In some embodiments, crystalline Compound 1 mono-HCl salt has an XRPD pattern comprising at least two peaks, in terms of 2-theta, selected from 10.0°±0.2°, 14.5°±0.2°, 19.9°±0.2°, 22.9°±0.2°, and 24.3°±0.2°.

In some embodiments, crystalline Compound 1 mono-HCl salt has an XRPD pattern comprising at least three peaks, in terms of 2-theta, selected from 10.0°±0.2°, 14.5°±0.2°, 19.90±0.2°, 22.9°±0.2°, and 24.3°±0.2°.

In some embodiments, crystalline Compound 1 mono-HCl salt has an XRPD pattern comprising at least four peaks, in terms of 2-theta, selected from 10.0°±0.2°, 14.5°±0.2°, 19.9°±0.2°, 22.9°±0.2°, and 24.3°±0.2°.

In some embodiments, crystalline Compound 1 mono-HCl salt has an XRPD pattern comprising peaks, in terms of 2-theta, at 10.0°±0.2°, 14.5°±0.2°, 19.9°±0.2°, 22.9°±0.2°, and 24.3°±0.2°.

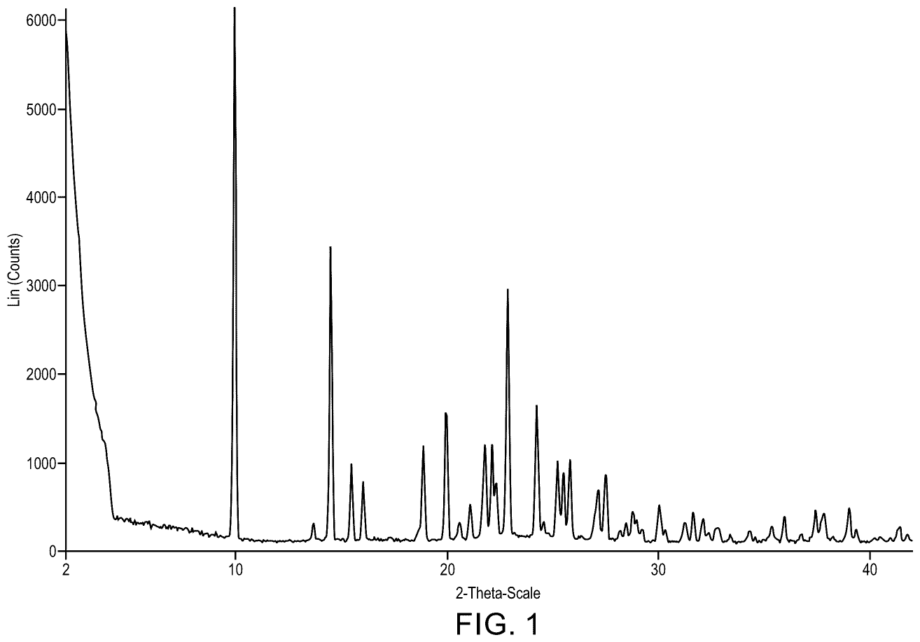

In some embodiments, crystalline Compound 1 mono-HCl salt has XRPD peaks substantially as shown in FIG. 1, wherein by “substantially” is meant that the reported peaks can vary by about ±0.2 °2-theta.

In some embodiments, crystalline Compound 1 mono-HCl salt has a crystalline structure that is monoclinic, space group P2₁.

In some embodiments, crystalline Compound 1 mono-HCl salt is prepared by precipitating the mono-HCl salt of Compound 1 from a solvent. In some embodiments, the solvent comprises an alcohol. In some embodiments, the alcohol comprises methanol, isopropanol, or both. In some embodiments, the solvent comprises acetonitrile. In some embodiments, the precipitating comprises evaporating a solution comprising Compound 1 and hydrochloric acid. For example, in some embodiments, crystalline Compound 1 mono-HCl salt was prepared by dissolving the mono-HCl salt of Compound 1 in methanol (10 vol.) and the solvent was removed by controlled evaporation.

### Crystalline Compound 1 Mono-HCl Salt (Single Crystal)

In some embodiments, crystalline Compound 1 mono-HCl salt is crystallized in the monoclinic crystal system with space group P2₁ having unit cell parameters where a is about 5.15 Å, b is about 12.78 Å, c is about 12.43 Å, and β is about 99.8°.

In some embodiments, crystalline Compound 1 mono-HCl salt is crystallized in the monoclinic crystal system with space group P2₁ having unit cell parameters where a is about 5.1 Å, b is about 12.8 Å, c is about 12.4 Å, and β is about 99.8°.

### Methods

Provided is a method of treating or preventing a 5-hydroxytryptamine (HT)_{2C} receptor-associated disorder in a patient in need thereof, wherein the method comprises administering to the patient crystalline Compound 1 HCl salt. In some embodiments, crystalline Compound 1 mono-HCl salt is administered. In some embodiments, a solid form preparation including crystalline Compound 1 mono-HCl salt is converted, shortly before administration, to a liquid form preparation. In some embodiments, the administration is twice daily. In some embodiments, the administration is three times daily.

Also provided is a method of treating or preventing epilepsy in a patient in need thereof, wherein the method comprises administering to the patient crystalline Compound 1 HCl salt. In some embodiments, crystalline Compound 1 mono-HCl salt is administered. In some embodiments, a solid form preparation including crystalline Compound 1 mono-HCl salt is converted, shortly before administration, to a liquid form preparation. In some embodiments, the administration is twice daily. In some embodiments, the administration is three times daily.

In some embodiments, the epilepsy is a focal epilepsy, a generalized epilepsy, or a combined generalized and focal epilepsy. In some embodiments, the epilepsy has one or more of a structural etiology, a genetic etiology, an infectious etiology, a metabolic etiology, and an immune etiology.

Also provided is a method of reducing severity of an epileptic seizure in a patient in need thereof, wherein the method comprises administering to the patient crystalline Compound 1 HCl salt. In some embodiments, crystalline Compound 1 mono-HCl salt is administered. In some embodiments, a solid form preparation including crystalline Compound 1 mono-HCl salt is converted, shortly before administration, to a liquid form preparation. In some embodiments, the administration is twice daily. In some embodiments, the administration is three times daily.

In some embodiments, the epileptic seizure is a focal seizure or a generalized seizure. In some embodiments, the epileptic seizure has one or more of a structural etiology, a genetic etiology, an infectious etiology, a metabolic etiology, and an immune etiology.

Also provided is a method of treating or preventing a seizure disorder in a patient in need thereof, wherein the method comprises administering to the patient crystalline Compound 1 HCl salt. In some embodiments crystalline Compound 1 mono-HCl salt is administered. In some embodiments, a solid form preparation including crystalline Compound 1 mono-HCl salt is converted, shortly before administration, to a liquid form preparation. In some embodiments, the administration is twice daily. In some embodiments, the administration is three times daily.

In some embodiments, the seizure disorder is a focal seizure disorder, a generalized seizure disorder, or combined generalized and focal seizure disorder. In some embodiments, the seizure disorder has one or more of a structural etiology, a genetic etiology, an infectious etiology, a metabolic etiology, and an immune etiology.

In some embodiments, the seizure disorder is selected from epilepsy, epilepsy with generalized tonic-clonic seizures, epilepsy with myoclonic absences, frontal lobe epilepsy, temporal lobe epilepsy, Landau-Kleffner Syndrome, Rasmussen's syndrome, Dravet syndrome, Doose syndrome (epilepsy with myoclonic atonic seizures (EM AS)), CDKL5 deficiency disorder (CDKL5 encephalopathy, or CDD), infantile spasms (West syndrome), juvenile myoclonic epilepsy (JME), vaccine-related encephalopathy, intractable childhood epilepsy (ICE), Lennox-Gastaut syndrome (LGS), Rett syndrome, Ohtahara syndrome (early infantile DEE, or EIDEE), childhood absence epilepsy, essential tremor, acute repetitive seizures, benign rolandic epilepsy, status epilepticus, refractory status epilepticus, super-refractory status epilepticus (SRSE). PCDH19 pediatric epilepsy, drug withdrawal induced seizures, alcohol withdrawal induced seizures, increased seizure activity and breakthrough seizures.

Also provided is a method of treating or preventing developmental and epileptic encephalopathy (DEE) in a patient in need thereof, wherein the method comprises administering to the patient crystalline Compound 1 HCl salt. In some embodiments, crystalline Compound 1 mono-HCl salt is administered. In some embodiments, a solid form preparation including crystalline Compound 1 mono-HCl salt is converted, shortly before administration, to a liquid form preparation. In some embodiments, the administration is twice daily. In some embodiments, the administration is three times daily.

In some embodiments, the DEE is chosen from epilepsy syndromes that involve a developmental impairment related to the underlying etiology (e.g., genetic mutation), frequent epileptiform activity, or both. In some embodiments, the DEE is epileptic. Epileptic encephalopathy (EE) can involve frequent epileptiform activity that contributes to an extent of cognitive and behavioral impartments that is more severe than what might be expected based on underlying etiology alone. In some embodiments, the DEE is developmental. Developmental encephalopathy (DE) can involve developmental consequences (e.g., developmental slowing or regression) that arise directly from underlying etiology, and may not involve frequent epileptic activity. In some embodiments, the DEE is both epileptic and developmental, in which developmental and epileptic factors both contribute to encephalopathy.

In some embodiments, the DEE is an early infantile DEE (EIDEE, or Ohtahara syndrome). In some embodiments, the EIDEE is an encephalopathy related to mutations in one or more of ARHGEF9, ARX, BRAT1, CASK, CDKL5, CYFIP2, DMXL2, GNAO1, KCNQ2, KCNT1, NECAP1, NSF, PCDH19, PIGA, PLCB1, PNKP, SCN2A, SCN8A, SIK1, SLC13A5, SLC25A22, SPTAN1, ST3GAL3, STXBP1, TBC1D24, WWOX, GLDC, AMT, GCSH, ALDH7A1, and PNPO. In some embodiments, the EIDEE is an encephalopathy associated with nonketotic hyperglycinemia (NKH; e.g., related to mutations in one or more of GLDC, AMT, GCSH), cortical dysplasia, and mitochondrial disorders.

In some embodiments, the EIDEE is a KCNQ2 encephalopathy, an SCN2A encephalopathy, an SCN8A encephalopathy, or a pyridoxine-dependent epilepsy (PDE).

In some embodiments, the DEE is an infantile epileptic spasms syndrome (IESS), which includes infants who present with epileptic spasms who do not fulfill the criteria for West syndrome. In some embodiments, the IESS is an encephalopathy related to mutations in one or more of AARS, ACADS ALG1, ALG13, ALPL, AMT, ARX, ATP22, ATP7A, CACNA1A, CACNA1C, CDKL5, CD99L2, CLCN4, CLCN6, CYFIP1, CYFIP2, DCX, DNM1, EEF1A2, FLNA, FOXG1, GABRE, GCSH, GLDC, GNAO1, GNB1, GPT2, GRIN2A, GRIN2B, HCN1, HEXA, IRF2BPL, KCNB1, KCNJ11, KCNQ2, KCNT1, KIF1A, KMT2D, LIS1 (also referred to as PAFAH1B1), MAGI2, MECP2, MED12, MEF2C, MMACHC MT-ND1, MYO18A, NEDD4L, NF1, NPRL3, NTRK2, PNKP, SCN2A, SCN8A, SCN10A, SETBP1, SETD5, SLC25A22, SLC35A2, SMARCA2, SPTAN1, STXBP1, TAF1, TBL1XR1, TCF4, TCF20, TSC1, TSC2, TUBA1A, UFC1, and WDR45. In some embodiments, the EISS is an encephalopathy related to 17p13.3 microdeletion, Xp22.13 microdeletion, 20q13.33 microdeletion, 9q33.3-34.11 microdeletion, 9p24.3-22.3 microdeletion, 5p12-11 microduplication, 3p25.3 microdeletion, 1p36.33 microdeletion, Xp22.11-21.3 microduplication, or 15q11.2 microduplication.

In some embodiments, the IESS is an encephalopathy associated with chromosome syndromes (e.g., 1p36 deletion syndrome, tetrasomy 12p, dup15q syndrome, trisomy 21 (Down syndrome)), NKH, organic acidemias, hypoxic ischemic encephalopathy (HE), neurofibromatosis, intracranial infection, brain injury secondary to neonatal hypoglycemia, intracranial hemorrhage (ICH), encephalomalacia, neuroglioma, focal brain lesion, pachygyria-lissencephaly, focal cortical dysplasia, heterotopia, polymicrogyria, schizencephaly, agenesis of the corpus callosum, intracranial hemangioma, Menkes disease, neurodegeneration with brain iron accumulation, methylmalonic acidemia (MMA), short-chain acyl-CoA dehydrogenase (SCAD) deficiency, lysosomal storage diseases, mitochondrial disorders, intra-uterine infection, GLUT-1 deficiency syndrome (hypoglycorrhachia), leukoencephalopathy, and hypophosphatasia.

In some embodiments, the IESS is tuberous sclerosis complex (TSC; associated with mutations in TSC1 or TSC2), an ARX encephalopathy, a CDKL5 encephalopathy (CDK5L deficiency disorder), or an STXBP1 encephalopathy.

In some embodiments, the DEE is chosen from Lennox-Gastaut syndrome, Dravet syndrome, Doose syndrome (EM AS), West syndrome (infantile spasms), Landau-Kleffner syndrome, and genetic disorders such as CDKL5 encephalopathy (CDK5L deficiency disorder) or CHD2 encephalopathy.

In some embodiments, the DEE is chosen from Ohtahara syndrome (EIDEE), Lennox-Gastaut syndrome, Dravet syndrome, Doose syndrome (EM AS), West syndrome (infantile spasms), Landau-Kleffner syndrome, tuberous sclerosis complex, CDKL5 encephalopathy (CDKL5 deficiency disorder), dup15q syndrome, SCN2A related epilepsies, SCN8A related epilepsies, KCNQ2 related epilepsies, KCNQ3 related epilepsies, Angelman syndrome, KCNT1 related epilepsies, SynGAP1 related epilepsies, Rett syndrome, PCDH19 epilepsy, ring 14 syndrome, ring 20 syndrome, CHD2 encephalopathy, early myoclonic encephalopathy, epilepsy of infancy with migrating focal seizures, and epileptic encephalopathy with continuous spike-wave.

Also provided is a method of treating or preventing a refractory epilepsy in a patient in need thereof, wherein the method comprises administering to the patient crystalline Compound 1 HCl salt. In some embodiments crystalline Compound 1 mono-HCl salt is administered. In some embodiments, a solid form preparation including crystalline Compound 1 mono-HCl salt is converted, shortly before administration, to a liquid form preparation. In some embodiments, the administration is twice daily. In some embodiments, the administration is three times daily.

In some embodiments, the patient has a comorbid condition, such as intellectual disability, autism spectrum disorder, and/or behavioral problems.

In some embodiments, the method provides improvement in at least one symptom selected from ataxia, gait impairment, speech impairment, vocalization, impaired cognition, abnormal motor activity, clinical seizure, subclinical seizure, hypotonia, hypertonia, drooling, mouthing behavior, aura, convulsions, repetitive movements, unusual sensations, frequency of seizures and severity of seizures.

In some embodiments, the administration results in an improvement in the frequency of convulsive/motor seizures and other seizure types. In some embodiments, the administration results in an improvement in one or more of the following:
frequency of observed countable motor seizures;
number of total seizures;
frequency of non-convulsive seizure;
number of episodes of status epilepticus;
frequency of use of rescue medication; and/or
number of countable motor seizure-free days.

In some embodiments, the administration results in an improvement in the Subject/Caregiver and Investigator Clinical Global Impression of Improvement (CGI-I), the Investigator Clinical Global Impression of Severity (CGI-S), and/or the 55-item Quality of Life in Childhood Epilepsy Questionnaire (QOLCE-55). In some embodiments, the administration results in at least a 1-point change from baseline in CGI-I and/or CGI-S.

In some embodiments, prior to administration, the patient had treatment resistant countable motor seizures with an average of ≥4 observed/countable motor seizures per 4-week period while on stable ASM treatment.

In some embodiments, the patient has a DEE but does not have Dravet syndrome or Lennox-Gastaut Syndrome.

In some embodiments wherein the patient has a DEE but does not have Dravet syndrome or Lennox-Gastaut Syndrome, the patient had:
a history of onset of unprovoked seizures at 5 years of age or earlier;
a history of developmental delay;
a history of combined focal and generalized seizure types or multiple generalized seizure types;
a history of slow or disorganized electroencephalogram; and/or
no history of idiopathic generalized seizures.

In some embodiments, the patient has Dravet syndrome.

In some embodiments wherein the patient has Dravet syndrome, prior to administration, the patient had:
onset of seizures between 3 and 12 months of age in an otherwise healthy infant; a history of seizures that were either generalized tonic-clonic or unilateral clonic or bilateral clonic;
normal initial development; and/or
a history of developmental delay.

In some embodiments wherein the patient has Dravet syndrome, prior to administration, the patient had:
an emergence of another seizure type;
prolonged exposure to warm temperatures-induced seizures and/or seizures that were associated with fevers due to illness or vaccines, hot baths, high levels of activity, and sudden temperature changes, and/or
seizures were induced by strong natural and/or fluorescent lighting.

In some embodiments wherein the patient has Dravet syndrome, prior to administration, the patient had genetic test results consistent with a diagnosis of Dravet syndrome.

In some embodiments the patient has Lennox-Gastaut Syndrome.

In some embodiments wherein the patient has Lennox-Gastaut Syndrome, prior to administration, the patient had:
a history of tonic seizures or tonic/atonic seizures;
more than 1 type of generalized seizure, including but not limited to generalized tonic-clonic, tonic-atonic, atonic, tonic, myoclonic, or drop seizures;
a history of seizure before 8 years of age.
a history of developmental delay.
a previous electroencephalogram reporting diagnostic criteria for Lennox-Gastaut Syndrome (abnormal inter-ictal electroencephalogram background activity accompanied by inter-ictal slow spike-and-wave pattern ≤2.5 hertz or interictal generalized paroxysmal fast activity); and/or
an average of ≥4 observed drop seizures per 4-week while on stable ASM treatment.

Also provided is a method of reducing likelihood of seizure-induced sudden unexpected death in epilepsy (SUDEP) in a patient in need thereof, wherein the method comprises administering to the patient crystalline Compound 1 HCl salt. In some embodiments crystalline Compound 1 mono-HCl salt is administered. In some embodiments, a solid form preparation including crystalline Compound 1 mono-HCl salt is converted, shortly before administration, to a liquid form preparation. In some embodiments, the administration is twice daily. In some embodiments, the administration is three times daily.

Also provided is a method of treating seizures in a patient suffering from a disease with which sudden unexpected death in epilepsy (SUDEP) can occur, wherein the method comprises administering to the patient crystalline Compound 1 HCl salt. In some embodiments crystalline Compound 1 mono-HCl salt is administered. In some embodiments, a solid form preparation including crystalline Compound 1 mono-HCl salt is converted, shortly before administration, to a liquid form preparation. In some embodiments, the administration is twice daily. In some embodiments, the administration is three times daily.

Also provided is a method of treating seizure-induced sudden unexpected death in epilepsy (SUDEP) in a patient suffering from a disease with which SUDEP can occur, wherein the method comprises administering to the patient crystalline Compound 1 HCl salt. In some embodiments crystalline Compound 1 mono-HCl salt is administered. In some embodiments, a solid form preparation including crystalline Compound 1 mono-HCl salt is converted, shortly before administration, to a liquid form preparation. In some embodiments, the administration is twice daily. In some embodiments, the administration is three times daily.

Also provided is a method of preventing or controlling seizure-induced sudden unexpected death in epilepsy (SUDEP) in a patient in need thereof, wherein the method comprises administering to the patient crystalline Compound 1 HCl salt. In some embodiments crystalline Compound 1 mono-HCl salt is administered. In some embodiments, a solid form preparation including crystalline Compound 1 mono-HCl salt is converted, shortly before administration, to a liquid form preparation. In some embodiments, the administration is twice daily. In some embodiments, the administration is three times daily.

In some embodiments the disease with which sudden unexpected death in epilepsy (SUDEP) can occur or is known to occur is a developmental and epileptic encephalopathy (DEE). In some embodiments, the disease with which sudden unexpected death in epilepsy (SUDEP) can occur or is known to occur is Dravet syndrome, Ohtahara syndrome, tuberous sclerosis complex (TSC), Angelman syndrome, myoclonic astatic epilepsy, Lennox-Gastaut syndrome, Doose syndrome (EM AS), West syndrome (infantile spasms), Prader-Willi syndrome, Landau-Kleffner syndrome, CDKL5 encephalopathy (CDKL5 deficiency disorder), dup15q syndrome, SCN2A related epilepsies, SCN8A related epilepsies, KCNQ2 related epilepsies, KCNQ3 related epilepsies, KCNT1 related epilepsies, SynGAP1 related epilepsies, Rett syndrome, PCDH19 epilepsy, ring 14 syndrome, ring 20 syndrome, CHD2 encephalopathy, early myoclonic encephalopathy, epilepsy of infancy with migrating focal seizures, or epileptic encephalopathy with continuous spike-wave. In some embodiments, the disease with which sudden unexpected death in epilepsy (SUDEP) can occur or is known to occur is Dravet syndrome. In some embodiments the disease with which sudden unexpected death in epilepsy (SUDEP) can occur or is known to occur is Lennox-Gastaut syndrome. In some embodiments, the patient is having more than three generalized tonic-clonic seizures per year. In some embodiments, the patient is not taking medications as prescribed. In some embodiments, the patient had an early age of epilepsy onset. In some embodiments, the patient has uncontrolled or frequent seizures. In some embodiments, administration results in a decreased incidence of seizures. In some embodiments, administration results in increased latency to seizures.

In some embodiments, the total daily dosage of crystalline Compound 1 mono-HCl salt is equivalent to about, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 mg of Compound 1. In some embodiments, the total daily dosage of crystalline Compound 1 mono-HCl salt is equivalent to about, 9, 12, 15, 18, 21, 24, 27, 30, 33, 36, 39, 42, 45, 48, 51, 54, 57, 60, 63, 66, 69, or 72 mg of Compound 1. In some embodiments, the total daily dosage of crystalline Compound 1 mono-HCl salt is equivalent to about 9 mg of Compound 1. In some embodiments, the total daily dosage of crystalline Compound 1 mono-HCl salt is equivalent to about 18 mg of Compound 1. In some embodiments, the total daily dosage of crystalline Compound 1 mono-HCl salt is equivalent to about 24 mg of Compound 1. In some embodiments, the total daily dosage of crystalline Compound 1 mono-HCl salt is equivalent to about 30 mg of Compound 1. In some embodiments, the total daily dosage of crystalline Compound 1 mono-HCl salt is equivalent to about 36 mg of Compound 1. In some embodiments, the total daily dosage of crystalline Compound 1 mono-HCl salt is equivalent to about 54 mg of Compound 1. In some embodiments, the total daily dosage of crystalline Compound 1 mono-HCl salt is equivalent to about 72 mg of Compound 1.

In some embodiments, crystalline Compound 1 mono-HCl salt is administered three times daily in a dosage equivalent to about 3 mg/dose of Compound 1. In some embodiments, crystalline Compound 1 mono-HCl salt is administered three times daily in a dosage equivalent to about 6 mg/dose of Compound 1. In some embodiments, crystalline Compound 1 mono-HCl salt is administered three times daily in a dosage equivalent to about 9 mg/dose of Compound 1. In some embodiments, crystalline Compound 1 mono-HCl salt is administered three times daily in a dosage equivalent to about 12 mg/dose of Compound 1. In some embodiments, crystalline Compound 1 mono-HCl salt is administered three times daily in a dosage equivalent to about 18 mg/dose of Compound 1. In some embodiments, crystalline Compound 1 mono-HCl salt is administered three times daily in a dosage equivalent to about 24 mg/dose of Compound 1.

In some embodiments, crystalline Compound 1 mono-HCl salt is administered twice daily in a dosage equivalent to about 3 mg/dose of Compound 1. In some embodiments, crystalline Compound 1 mono-HCl salt is administered twice daily in a dosage equivalent to about 6 mg/dose of Compound 1. In some embodiments, crystalline Compound 1 mono-HCl salt is administered twice daily in a dosage equivalent to about 9 mg/dose of Compound 1. In some embodiments, crystalline Compound 1 mono-HCl salt is administered twice daily in a dosage equivalent to about 12 mg/dose of Compound 1. In some embodiments, crystalline Compound 1 mono-HCl salt is administered twice daily in a dosage equivalent to about 15 mg/dose of Compound 1. In some embodiments, crystalline Compound 1 mono-HCl salt is administered twice daily in a dosage equivalent to about 18 mg/dose of Compound 1. In some embodiments, crystalline Compound 1 mono-HCl salt is administered twice daily in a dosage equivalent to about 24 mg/dose of Compound 1.

In some embodiments, crystalline Compound 1 mono-HCl salt is administered in an amount based on the body weight of the patient.

In some embodiments, crystalline Compound 1 mono-HCl salt is administered via a titration scheme that comprises the up-titration of crystalline Compound 1 mono-HCl salt until an optimized dosage is administered.

In some embodiments, the titration scheme comprises administering crystalline Compound 1 mono-HCl salt at an initial dosage equivalent to about 0.11 mg/kg of Compound 1 three times daily and, provided that the patient tolerates the initial dosage and that the patient has not had an adequate response, increasing the dosage.

In some embodiments, the patient is less than or equal to 2 years of age and is administered crystalline Compound 1 mono-HCl salt at an initial dosage equivalent to about 0.11 mg/kg of Compound 1 three times daily.

In some embodiments, the increased dosage is equivalent to about 0.17 mg/kg of Compound 1 three times daily.

In some embodiments, if the patient does not tolerate the increased dosage, the optimized dosage is the initial dosage.

In some embodiments, if the patient tolerates the increased dosage and if the patient has had an adequate response, the optimized dosage is the increased dosage.

In some embodiments, the titration scheme comprises further increasing the dosage, provided that the patient tolerates the increased dosage and that the patient has not had an adequate response.

In some embodiments, the further increased dosage is equivalent to about 0.24 mg/kg Compound 1 free base three times daily.

In some embodiments, if the patient tolerates the further increased dosage and if the patient has had an adequate response, the optimized dosage is the further increased dosage (or maintenance dose selected from 0.11 mg/kg, 0.17 mg/kg or 0.24 mg/kg.).

In some embodiments, the method further comprises administering the optimized dosage of crystalline Compound 1 mono-HCl salt to the patient.

In some embodiments, the dosage of crystalline Compound 1 mono-HCl salt is down-titrated to address an observed side effect. In some embodiments, the dosage of crystalline Compound 1 mono-HCl salt is down-titrated to minimize the risk of a withdrawal induced side effect.

In some embodiments, the dosage of crystalline Compound 1 mono-HCl salt is gradually increased, i.e., up-titrated, to achieve the highest maintenance dose based on tolerability.

In some embodiments, the patient is less than 2 years of age. In some embodiments, the patient is less than 2 years of age and the maintenance dose is equivalent to up to about 0.3 mg/kg Compound 1 free base three times daily.

In some embodiments, the patient is an infant (29 days to less than 2 years). In some embodiments, the patient is an infant (29 days to less than 2 years) and is administered crystalline Compound 1 mono-HCl salt at a dosage equivalent to from about 0.03 to about 0.05 mg/kg Compound 1 free base three times daily as the initial dose.

In some embodiments, the patient is less than 2 years of age and is administered crystalline Compound 1 mono-HCl salt at a dosage equivalent to about 0.03 mg/kg Compound 1 free base three times daily. In some embodiments, the patient is less than 2 years of age and is administered crystalline Compound 1 mono-HCl salt at a dosage equivalent to about 0.05 mg/kg Compound 1 free base three times daily as the initial dose.

In some embodiments, the patient is an infant (29 days to less than 2 years) and has body weight of approximately 3 to 10 kg. In some embodiments, the patient is an infant boy and has a body weight between about 10.6 kg and about 15.2 kg. In some embodiments, the patient is an infant female and has a body weight between about and 10.2 kg and 14.6 kg.

In some embodiments, the patient is a child (2 years to less than 12 years).

In some embodiments, the patient is an adolescent (12 years to 21 years (up to but not including the 22^{nd} birthday)).

In some embodiments, the patient is an adult (22 years or older).

In some embodiments, the patient is ≤40 kg and crystalline Compound 1 mono-HCl salt is administered at a dose equivalent to Compound 1 free base three times daily (TID) according to the following schedule:

| Age | | Initial dose | | Up-titration | | Up- titration | Up- titration | Up- titration |
|---|---|---|---|---|---|---|---|---|
| stratification* | | (TID) | | (TID) | | (TID) | (TID) | (TID) |
| 0-<2 | years | 0.03-0.05 | mg/kg | 0.07-0.9 | mg/kg | 0.11 mg/kg | 0.17 mg/kg | 0.24 mg/kg |
| 2-5 | years | 0.11 | mg/kg | 0.17 | mg/kg | 0.24 mg/kg | 0.30 mg/kg | − |
| 6-11 | years | 0.11 | mg/kg | 0.17 | mg/kg | 0.24 mg/kg | 0.30 mg/kg | − |
| 12 < 18 | years | 0.11 | mg/kg | 0.17 | mg/kg | 0.24 mg/kg | 0.30 mg/kg | = |

Bodyweight based dosing for ≤40 kg and fixed dosing if >40 kg bodyweight

In some embodiments, administration of crystalline Compound 1 HCl salt can achieve one or more pharmacokinetic characteristics, including a geometric mean steady-state C_{trough} in CSF, a geometric mean steady-state C_{trough} in plasma, or both. In some embodiments, the administration results in a ratio of a geometric mean steady-state C_{trough} of Compound 1 in CSF to a geometric mean steady-state C_{trough} of Compound 1 in plasma (CSF/P C_{trough}) of at least about 1.4. In some embodiments, the administration results in a CSF/P C_{trough} of about 1.4 to about 3, about 1.4 to about 2.5, about 1.4 to about 2, about 1.5 to about 3, about 1.5 to about 2.5, or about 1.5 to about 2. In some embodiments, the administration results in a CSF/P C_{trough} of about 1.5, about 1.7, or about 1.9.

In some embodiments, the administration results in a geometric mean steady-state C_{trough} of Compound 1 in CSF of about 3 ng/mL to about 15 ng/mL, for example, about 3 ng/mL to about 12 ng/mL, about 3 ng/mL to about 10 ng/mL, about 4 ng/mL to about 15 ng/mL, about 4 ng/mL to about 12 nm/mL, about 4 ng/mL to about 10 ng/mL, about 5 ng/mL to about 15 ng/mL, about 5 ng/mL to about 12 ng/mL, about 5 ng/L to about 10 ng/mL. In some embodiments, the administration results in a geometric mean steady-state C_{trough} of Compound 1 in CSF of about 6.5 ng/mL, about 7 ng/mL, or about 7.7 ng/mL.

In some embodiments, the administration results in a geometric mean steady-state C_{trough} of Compound 1 in plasma of about 2 ng/mL to about 12 ng/mL, for example, about 2 ng/mL to about 10 ng/mL, about 2 ng/mL to about 9 ng/mL, about 3 ng/mL to about 12 ng/mL, about 3 ng/mL to about 10 ng/mL, about 3 ng/mL to about 9 ng/mL, about 4 ng/mL to about 12 ng/mL, about 4 ng/mL to about 10 ng/mL, or about 4 ng/mL to about 9 ng/mL. In some embodiments, the administration results in a geometric mean steady-state C_{trough} of Compound 1 in plasma of about 4.0 ng/mL, about 4.4 ng/mL, about 4.9 ng/mL, about 5.2 ng/mL, about 5.6 ng/mL, about 6.5 ng/mL, about 7.1 ng/mL, or about 7.4 ng/mL.

In some embodiments, crystalline Compound 1 mono-HCl salt is administered via a titration scheme that includes the up-titration of Compound 1 mono-HCl salt until an optimized dosage is administered.

In some embodiments, the total daily dosage of crystalline Compound 1 mono-HCl salt is titrated to a dosage equivalent to about 9 mg of Compound 1. In some embodiments, the total daily dosage of crystalline Compound 1 mono-HCl salt is titrated to a dosage equivalent to about 18 mg of Compound 1. In some embodiments, the total daily dosage of crystalline Compound 1 mono-HCl salt is titrated to a dosage equivalent to about 24 mg of Compound 1. In some embodiments, the total daily dosage of crystalline Compound 1 mono-HCl salt is titrated to a dosage equivalent to about 30 mg of Compound 1. In some embodiments, the total daily dosage of crystalline Compound 1 mono-HCl salt is titrated to a dosage equivalent to about 36 mg of Compound 1. In some embodiments, the total daily dosage of crystalline Compound 1 mono-HCl salt is titrated to a dosage equivalent to about 54 mg of Compound 1. In some embodiments, the total daily dosage of crystalline Compound 1 mono-HCl salt is titrated to a dosage equivalent to about 72 mg of Compound 1.

In some embodiments, the total daily dosage of crystalline Compound 1 mono-HCl salt is down-titrated to a dosage equivalent to about, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, or 17 mg of Compound 1. In some embodiments, the total daily dosage of crystalline Compound 1 mono-HCl salt is down-titrated to a dosage equivalent to about 54 mg of Compound 1. In some embodiments, the total daily dosage of crystalline Compound 1 mono-HCl salt is down-titrated to a dosage equivalent to about 36 mg of Compound 1. In some embodiments, the total daily dosage of crystalline Compound 1 mono-HCl salt is down-titrated to a dosage equivalent to about 30 mg of Compound 1. In some embodiments, the total daily dosage of crystalline Compound 1 mono-HCl salt is down-titrated to a dosage equivalent to about 24 mg of Compound 1. In some embodiments, the total daily dosage of crystalline Compound 1 mono-HCl salt is down-titrated to a dosage equivalent to about 18 mg of Compound 1. In some embodiments, the total daily dosage of crystalline Compound 1 mono-HCl salt is down-titrated to a dosage equivalent to about 12 mg of Compound 1. In some embodiments, the total daily dosage of crystalline Compound 1 mono-HCl salt is down-titrated to a dosage equivalent to about 9 mg of Compound 1.

In some embodiments, the increasing of the dosage of crystalline Compound 1 mono-HCl salt in increments equivalent to about 3 mg/dose of Compound 1 about every 1, 2, 3, 4, or 5 days until the optimized dosage is administered. In some embodiments, the optimized dosage is equivalent to about 3 mg/dose of Compound 1. In some embodiments, the optimized dosage is equivalent to about 6 mg/dose of Compound 1. In some embodiments, the optimized dosage is equivalent to about 9 mg/dose of Compound 1. In some embodiments, the optimized dosage is equivalent to about 12 mg/dose of Compound 1. In some embodiments, the optimized dosage is equivalent to about 15 mg/dose of Compound 1. In some embodiments, the optimized dosage is equivalent to about 18 mg/dose of Compound 1.

In some embodiments, the titration scheme comprises prescribing and/or administering crystalline Compound 1 mono-HCl salt at an initial dosage equivalent to about 6 mg/dose of Compound 1 and, provided that the patient tolerates the initial dosage, and that the patient has not had an adequate response, increasing the dosage. In some embodiments, the titration scheme comprises prescribing and/or administering crystalline Compound 1 mono-HCl salt two or three times daily at an initial dosage equivalent to about 6 mg/dose of Compound 1 for about 1, 2, 3, 4, or 5 days and, provided that the patient tolerates the initial dosage, and that the patient has not had an adequate response, increasing the dosage. In some embodiments, the titration scheme comprises prescribing and/or administering crystalline Compound 1 mono-HCl salt at an initial dosage equivalent to about 6 mg/dose of Compound 1 for about 2 days and, provided that the patient tolerates the initial dosage, and that the patient has not had an adequate response, increasing the dosage.

In some embodiments, the increased dosage is equivalent to about 9 mg/dose of Compound 1. In some embodiments, the increased dosage is equivalent to about 12 mg/dose of Compound 1. In some embodiments, the increased dosage is equivalent to about 15 mg/dose of Compound 1. In some embodiments, the increased dosage is equivalent to about 18 mg/dose of Compound 1.

In some embodiments, the titration scheme further comprises administering crystalline Compound 1 mono-HCl salt at the increased dosage for about two days. In some embodiments, the titration scheme further comprises administering crystalline Compound 1 mono-HCl salt at the increased dosage for about five days.

In some embodiments, if the patient does not tolerate the increased dosage, the optimized dosage is the initial dosage.

In some embodiments, if the patient tolerates the increased dosage and if the patient has had an adequate response, the optimized dosage is the increased dosage.

In some embodiments, the titration scheme comprises further increasing the dosage, provided that the individual tolerates the increased dosage, and that the individual has not had an adequate response. In some embodiments, the further increased dosage is equivalent to about 12 mg/dose of Compound 1. In some embodiments, the further increased dosage is equivalent to about 15 mg/dose of Compound 1. In some embodiments, the further increased dosage is equivalent to about 18 mg/dose of Compound 1.

In some embodiments, the titration scheme further comprises administering crystalline Compound 1 mono-HCl salt at the further increased dosage for about five days.

In some embodiments, if the patient does not tolerate the further increased dosage, the optimized dosage is the increased dosage.

In some embodiments, if the patient tolerates the further increased dosage and if the patient has had an adequate response, the optimized dosage is the further increased dosage.

In some embodiments, the titration scheme further comprises administering the optimized dosage of crystalline Compound 1 mono-HCl salt to the patient.

In some embodiments, if the patient tolerates the increased dosage and if the patient has not had an adequate response, the method further comprises increasing the dosage.

In some embodiments, the titration scheme further comprises administering the optimized dosage of crystalline Compound 1 mono-HCl salt to the patient.

In some embodiments, for example, when the patient does not tolerate the increased dosage of crystalline Compound 1 mono-HCl salt the titration scheme further comprises down-titration of crystalline Compound 1, or a pharmaceutically acceptable salt thereof. In some embodiments, the down-titration comprises reducing the dosage of crystalline Compound 1 mono-HCl salt being administered to the patient by an increment equivalent to about, 1, 2, 3, 4, 5, 6, 7, 8, 9, 10, 11, or 12 mg/dose of Compound 1. In some embodiments, the down-titration scheme comprises reducing the dosage of crystalline Compound 1 mono-HCl salt being administered to the patient once. In some embodiments, the down-titration scheme comprises reducing the dosage of crystalline Compound 1 mono-HCl salt being administered to the patient more than once. In some embodiments, the down-titration scheme comprises reducing the dosage of crystalline Compound 1 mono-HCl salt being administered to the patient in increments equivalent to about 3 mg/dose of Compound 1 about every 1, 2, 3, 4, or 5 days until the patient is no longer being administered crystalline Compound 1, or a pharmaceutically acceptable salt thereof.

In some embodiments, the dosage of crystalline Compound 1 mono-HCl salt is down-titrated to address an observed side effect. In some embodiments, the dosage of crystalline Compound 1 mono-HCl salt is down-titrated to minimize the risk of a withdrawal induced side effect.

In some embodiments, the patient is also being administered an antiepileptic drug or antiseizure medicine. In some embodiments, the patient is also being administered an antiepileptic drug effective in suppressing interictal epileptiform discharges (e.g, benzodiazepines, valproic acid, and lamotrigine). In some embodiments, the patient is also being administered an immunomodulatory therapy (e.g., corticosteroids, intravenous immunoglobulin [IVIG], plasmapheresis). In some embodiments, the patient is also being administered a ketogenic diet.

In some embodiments, the patient is also being administered a CYP enzyme inhibitor, and no adjustments to the dosage of crystalline Compound 1 mono-HCl salt are made as compared to a corresponding patient not being administered the CYP enzyme inhibitor. In some embodiments, the CYP enzyme inhibitor is a moderate CYP enzyme inhibitor or a strong CYP enzyme inhibitor. In some embodiments, the CYP enzyme inhibitor is an antiseizure medicine. In some embodiments, the CYP enzyme inhibitor is fenfluramine, carbamazepine, clobazam, cannabidiol, felbamate, phenobarbital, or phenytoin. In some embodiments, the CYP enzyme inhibitor is a substrate for CYP2D6, CYP3A4, CYP2C19, or CYP2C9.

In some embodiments, the patient is also being administered a P-glycoprotein (P-gp) inhibitor, and no dose adjustments to the dosage of crystalline Compound 1 mono-HCl salt are made as compared to a corresponding patient not being administered the P-gp inhibitor. In some embodiments, the P-gp inhibitor is a P-gp-mediated efflux or renal transporter.

In some embodiments, crystalline Compound 1 mono-HCl salt is administered as a raw or pure chemical, for example as a powder in capsule formulation.

In some embodiments, crystalline Compound 1 mono-HCl salt is formulated as a pharmaceutical composition further comprising one or more pharmaceutically acceptable carriers.

### Compositions

Provided are compositions comprising crystalline Compound 1 HCl salt. In some embodiments, the composition includes crystalline Compound 1 mono-HCl salt and a pharmaceutically acceptable carrier.

Pharmaceutical compositions may be prepared by any suitable method, for example by uniformly mixing the active compound(s) with finely divided solid carriers, in the required proportions and then, if necessary, forming the resulting mixture into a desired shape.

Conventional excipients, such as binding agents, fillers, acceptable wetting agents, tableting lubricants and disintegrants may be used in tablets and capsules for oral administration. The forms described herein can be formulated into pharmaceutical compositions using techniques well known to those in the art. Suitable pharmaceutically acceptable carriers, outside those mentioned herein, are known in the art; for example, see Remington, The Science and Practice of Pharmacy, 20^{th} Edition, 2000, Lippincott Williams & Wilkins, (Editors: Gennaro et al.)

In some embodiments, crystalline Compound 1 HCl salt is formulated in a manner suitable for oral administration.

For oral administration, the pharmaceutical composition may be in the form of, for example, a tablet or capsule. The pharmaceutical composition is preferably made in the form of a dosage unit containing a particular amount of the active ingredient. Examples of such dosage units are capsules, tablets, powders, granules, or suspensions,

In powders, the carrier is a finely divided solid which is in a mixture with the finely divided active component.

In tablets, the active component is mixed with the carrier having the necessary binding capacity in suitable proportions and compacted to the desired shape and size.

For oral administration, the pharmaceutical composition may be in the form of suitable for administration via gastrostomy tube or percutaneous endoscopic gastrostomy tube.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets or capsules. Also, the unit dosage form can be a capsule or tablet itself, or it can be the appropriate number of any of these in packaged form.

Also provided are solid form preparations which are intended to be converted, shortly before use, to liquid form preparations for oral administration. Such liquid forms include solutions, suspensions, and emulsions.

Also provided is an oral solution comprising crystalline Compound 1 mono-HCl Salt as an active ingredient and at least one pharmaceutically acceptable aqueous carrier.

Further embodiments include the embodiments disclosed in the following Examples, which is not to be construed as limiting in any way.

### FURTHER EMBODIMENTS OF THE INVENTION

The invention further relates to the following embodiments (E1-E42):
E1. The mono-HCl salt of (R)—N-(2,2-difluoroethyl)-7-methyl-1,2,3,4,6,7-hexahydro-[1,4]diazepino[6,7,1-hi]indole-8-carboxamide (Compound 1) which is substantially crystalline.
E2. The crystalline salt of E1, having an XRPD pattern comprising a peak, in terms of 2-theta, at 10.0°±0.2°.
E3. The crystalline salt of any one of E1-E2, having an XRPD pattern comprising peaks, in terms of 2-theta, at 10.0°±0.2°, 14.5°±0.2°, and 22.9°±0.2°.
E4. The crystalline salt of any one of E1-E3, having an XRPD pattern further comprising peaks, in terms of 2-theta, at 19.9°±0.2° and 24.3°±0.2°.
E5. The crystalline salt of any one of E1-E4, having an XRPD pattern with peaks substantially as shown in FIG. 1.
E6. A pharmaceutical composition comprising the crystalline salt of any one of E1-E5, and a pharmaceutically acceptable carrier.
E7. A method of treating or preventing a 5-hydroxytryptamine (HT)_{2C} receptor-associated disorder in a patient in need thereof, wherein the method comprises administering to the patient the crystalline salt of any one of E1-E5 or the composition of E6.
E8. A method of treating or preventing epilepsy in a patient in need thereof, wherein the method comprises administering to the patient the crystalline salt of any one of E1-E5 or the composition of E6.
E9. A method of reducing severity of an epileptic seizure in a patient in need thereof, wherein the method comprises administering to the patient the crystalline salt of any one of E1-E5 or the composition of E6.
E10. A method of reducing the frequency of epileptic seizures in a patient in need thereof, wherein the method comprises administering to the patient the crystalline salt of any one of E1-E5 or the composition of E6.
E11. A method of treating or preventing a seizure disorder in a patient in need thereof, wherein the method comprises administering to the patient the crystalline salt of any one of E1-E5 or the composition of E6.
E12. The method of E11, wherein the seizure disorder is selected from epilepsy, epilepsy with generalized tonic-clonic seizures, epilepsy with myoclonic absences, frontal lobe epilepsy, temporal lobe epilepsy, Landau-Kleffner Syndrome, Rasmussen's syndrome, Dravet syndrome, Doose syndrome (epilepsy with myoclonic atonic seizures (EM AS)), CDKL5 deficiency disorder (CDKL5 encephalopathy, or CDD), infantile spasms (West syndrome), juvenile myoclonic epilepsy (JME), vaccine-related encephalopathy, intractable childhood epilepsy (ICE), Lennox-Gastaut syndrome (LGS), Rett syndrome, Ohtahara syndrome (early infantile DEE, EIDEE), childhood absence epilepsy, essential tremor, acute repetitive seizures, benign rolandic epilepsy, status epilepticus, refractory status epilepticus, super-refractory status epilepticus (SRSE), PCDH19 pediatric epilepsy, drug withdrawal induced seizures, alcohol withdrawal induced seizures, increased seizure activity and breakthrough seizures.
E13. A method of treating developmental and epileptic encephalopathy (DEE) in a patient in need thereof, wherein the method comprises administering to the patient the crystalline salt of any one of E1-E5 or the composition of E6.
E14. The method of E13, wherein the DEE is selected from Lennox-Gastaut syndrome, Dravet syndrome, Doose syndrome (EM AS), West syndrome (infantile spasms), Landau-Kleffner syndrome, and genetic disorders such as CDKL5 encephalopathy (CDK5L deficiency disorder) or CHD2 encephalopathy.
E15. The method of E13, wherein the DEE is selected from Ohtahara syndrome (EIDEE), Lennox-Gastaut syndrome, Dravet syndrome, Doose syndrome (EM AS), West syndrome (infantile spasms), Landau-Kleffner syndrome, tuberous sclerosis complex, CDKL5 encephalopathy (CDKL5 deficiency disorder), dup15q syndrome, SCN2A related epilepsies, SCN8A related epilepsies, KCNQ2 related epilepsies, KCNQ3 related epilepsies, Angelman syndrome, KCNT1 related epilepsies, SynGAP1 related epilepsies, Rett syndrome, PCDH19 epilepsy, ring 14 syndrome, ring 20 syndrome, CHD2 encephalopathy, early myoclonic encephalopathy, epilepsy of infancy with migrating focal seizures, and epileptic encephalopathy with continuous spike-wave.
E16. A method of treating a refractory epilepsy in a patient in need thereof, wherein the method comprises administering to the patient the crystalline salt of any one of E1-E5 or the composition of E6.
E17. A process for preparing the crystalline salt of any one of E1-E5, comprising precipitating the mono-HCl salt of Compound 1 from a solvent.
E18. The process of E17, wherein the solvent comprises an alcohol.
E19. The process of E18, wherein the alcohol is methanol or isopropanol.
E20. The process of E17, wherein the solvent comprises acetonitrile.
E21. The process of E17, wherein the precipitating comprises evaporating a solution comprising Compound 1 and hydrochloric acid.
E22. The mono-HCl salt of (R)—N-(2,2-difluoroethyl)-7-methyl-1,2,3,4,6,7-hexahydro-[1,4]diazepino[6,7,1-hi]indole-8-carboxamide (Compound 1), prepared by the process of any one of E17-E21.
E23. The crystalline salt of any one of E1-E5 or the composition of E6 for use in treating or preventing a 5-hydroxytryptamine (HT)_{2C} receptor-associated disorder in a patient in need thereof, wherein the use comprises administering to the patient the crystalline salt of any one of E1-E5 or the composition of E6.
E24. The crystalline salt of any one of E1-E5 or the composition of E6 for use in treating or preventing epilepsy in a patient in need thereof, wherein the use comprises administering to the patient the crystalline salt of any one of E1-E5 or the composition of E6.
E25. The crystalline salt of any one of E1-E5 or the composition of E6 for use in reducing severity of an epileptic seizure in a patient in need thereof, wherein the use comprises administering to the patient the crystalline salt of any one of E1-E5 or the composition of E6.
E26. The crystalline salt of any one of E1-E5 or the composition of E6 for use in reducing the frequency of epileptic seizures in a patient in need thereof, wherein the use comprises administering to the patient the crystalline salt of any one of E1-E5 or the composition of E6.
E27. The crystalline salt of any one of E1-E5 or the composition of E6 for use in treating or preventing a seizure disorder in a patient in need thereof, wherein the use comprises administering to the patient the crystalline salt of any one of E1-E5 or the composition of E6.
E28. The crystalline salt or composition for use of E27, wherein the seizure disorder is selected from epilepsy, epilepsy with generalized tonic-clonic seizures, epilepsy with myoclonic absences, frontal lobe epilepsy, temporal lobe epilepsy, Landau-Kleffner Syndrome, Rasmussen's syndrome, Dravet syndrome, Doose syndrome (epilepsy with myoclonic atonic seizures (EM AS)), CDKL5 deficiency disorder (CDKL5 encephalopathy, or CDD), infantile spasms (West syndrome), juvenile myoclonic epilepsy (JME), vaccine-related encephalopathy, intractable childhood epilepsy (ICE), Lennox-Gastaut syndrome (LGS), Rett syndrome, Ohtahara syndrome (early infantile DEE, EIDEE), childhood absence epilepsy, essential tremor, acute repetitive seizures, benign rolandic epilepsy, status epilepticus, refractory status epilepticus, super-refractory status epilepticus (SRSE), PCDH19 pediatric epilepsy, drug withdrawal induced seizures, alcohol withdrawal induced seizures, increased seizure activity and breakthrough seizures.
E29. The crystalline salt of any one of E1-E5 or the composition of E6 for use in treating developmental and epileptic encephalopathy (DEE) in a patient in need thereof, wherein the use comprises administering to the patient the crystalline salt of any one of E1-E5 or the composition of E6.
E30. The crystalline salt or composition for use of E29, wherein the DEE is selected from Lennox-Gastaut syndrome, Dravet syndrome, Doose syndrome (EM AS), West syndrome (infantile spasms), Landau-Kleffner syndrome, and genetic disorders such as CDKL5 encephalopathy (CDK5L deficiency disorder) or CHD2 encephalopathy.
E31. The crystalline salt or composition for use of E29, wherein the DEE is selected from Ohtahara syndrome (EIDEE), Lennox-Gastaut syndrome, Dravet syndrome, Doose syndrome (EM AS), West syndrome (infantile spasms), Landau-Kleffner syndrome, tuberous sclerosis complex, CDKL5 encephalopathy (CDKL5 deficiency disorder), dup15q syndrome, SCN2A related epilepsies, SCN8A related epilepsies, KCNQ2 related epilepsies, KCNQ3 related epilepsies, Angelman syndrome, KCNT1 related epilepsies, SynGAP1 related epilepsies, Rett syndrome, PCDH19 epilepsy, ring 14 syndrome, ring 20 syndrome, CHD2 encephalopathy, early myoclonic encephalopathy, epilepsy of infancy with migrating focal seizures, and epileptic encephalopathy with continuous spike-wave.
E32. The crystalline salt of any one of E1-E5 or the composition of E6 for use in treating a refractory epilepsy in a patient in need thereof, wherein the use comprises administering to the patient the crystalline salt of any one of E1-E5 or the composition of E6.
E33. Use of the crystalline salt of any one of E1-E5 or the composition of E6 for the manufacture of a medicament for treating or preventing a 5-hydroxytryptamine (HT)_{2C} receptor-associated disorder in a patient in need thereof, wherein the use comprises administering to the patient the crystalline salt of any one of E1-E5 or the composition of E6.
E34. Use of the crystalline salt of any one of E1-E5 or the composition of E6 for the manufacture of a medicament for treating or preventing epilepsy in a patient in need thereof, wherein the use comprises administering to the patient the crystalline salt of any one of E1-E5 or the composition of E6.
E35. Use of the crystalline salt of any one of E1-E5 or the composition of E6 for the manufacture of a medicament for reducing severity of an epileptic seizure in a patient in need thereof, wherein the use comprises administering to the patient the crystalline salt of any one of E1-E5 or the composition of E6.
E36. Use of the crystalline salt of any one of E1-E5 or the composition of E6 for the manufacture of a medicament for reducing the frequency of epileptic seizures in a patient in need thereof, wherein the use comprises administering to the patient the crystalline salt of any one of E1-E5 or the composition of E6.
E37. Use of the crystalline salt of any one of E1-E5 or the composition of E6 for the manufacture of a medicament for treating or preventing a seizure disorder in a patient in need thereof, wherein the use comprises administering to the patient the crystalline salt of any one of E1-E5 or the composition of E6.
E38. The use according to E37, wherein the seizure disorder is selected from epilepsy, epilepsy with generalized tonic-clonic seizures, epilepsy with myoclonic absences, frontal lobe epilepsy, temporal lobe epilepsy, Landau-Kleffner Syndrome, Rasmussen's syndrome, Dravet syndrome, Doose syndrome (epilepsy with myoclonic atonic seizures (EM AS)), CDKL5 deficiency disorder (CDKL5 encephalopathy, or CDD), infantile spasms (West syndrome), juvenile myoclonic epilepsy (JME), vaccine-related encephalopathy, intractable childhood epilepsy (ICE), Lennox-Gastaut syndrome (LGS), Rett syndrome, Ohtahara syndrome (early infantile DEE, EIDEE), childhood absence epilepsy, essential tremor, acute repetitive seizures, benign rolandic epilepsy, status epilepticus, refractory status epilepticus, super-refractory status epilepticus (SRSE), PCDH19 pediatric epilepsy, drug withdrawal induced seizures, alcohol withdrawal induced seizures, increased seizure activity and breakthrough seizures.
E39. Use of the crystalline salt of any one of E1-E5 or the composition of E6 for the manufacture of a medicament for treating developmental and epileptic encephalopathy (DEE) in a patient in need thereof, wherein the use comprises administering to the patient the crystalline salt of any one of E1-E5 or the composition of E6.
E40. The use according to E39, wherein the DEE is selected from Lennox-Gastaut syndrome, Dravet syndrome, Doose syndrome (EM AS), West syndrome (infantile spasms), Landau-Kleffner syndrome, and genetic disorders such as CDKL5 encephalopathy (CDK5L deficiency disorder) or CHD2 encephalopathy.
E41. The use according to E39, wherein the DEE is selected from Ohtahara syndrome (EIDEE), Lennox-Gastaut syndrome, Dravet syndrome, Doose syndrome (EM AS), West syndrome (infantile spasms), Landau-Kleffner syndrome, tuberous sclerosis complex, CDKL5 encephalopathy (CDKL5 deficiency disorder), dup15q syndrome, SCN2A related epilepsies, SCN8A related epilepsies, KCNQ2 related epilepsies, KCNQ3 related epilepsies, Angelman syndrome, KCNT1 related epilepsies, SynGAP1 related epilepsies, Rett syndrome, PCDH19 epilepsy, ring 14 syndrome, ring 20 syndrome, CHD2 encephalopathy, early myoclonic encephalopathy, epilepsy of infancy with migrating focal seizures, and epileptic encephalopathy with continuous spike-wave.
E42. Use of the crystalline salt of any one of E1-E5 or the composition of E6 for the manufacture of a medicament for treating a refractory epilepsy in a patient in need thereof, wherein the use comprises administering to the patient the crystalline salt of any one of E1-E5 or the composition of E6.

### EXAMPLES

### Example 1. Instruments and Data Collection Parameters

### X-Ray Powder Diffraction (XRPD)

XRPD analysis was carried out on a Bruker AXS D8 Advance, as discussed below.

XRPD was measured at ambient conditions in a Bruker AXS D8 Advance using Cu Kα radiation (40 kV, 40 mA) and a θ-2θ goniometer fitted with a Ge monochromator. The samples were measured in the range of 2° to 42° 2θ, at a step size of 0.05° 2θ using a Lynxeye Detector. The collection time was 0.5 s/step (total collection time: 6.40 min). The software used for data collection and analysis was Diffrac Plus XRD Commander and Diffrac Plus EVA, respectively.

### Single Crystal X-Ray Diffraction (SCXRD)

SCXRD analysis was carried out on a Rigaku Oxford Diffraction Supernova Dual Source, Cu at Zero, Atlas CCD diffractometer equipped with an Oxford Cryosystems Cobra cooling device. The data were collected using Cu Kα radiation as shown in Table 1, below.

TABLE 1
SCXRD Radiation Parameters

| | | |
|---|---|---|
| Temperature | 100(2) | K |
| Wavelength | 1.54184 | Å |

Structures were solved and refined using the Bruker AXS SHELXTL suite or the OLEX crystallographic software. Unless otherwise stated, hydrogen atoms attached to carbon were placed geometrically and allowed to refine with a riding isotropic displacement parameter. Hydrogen atoms attached to a heteroatom were located in a difference Fourier synthesis and were allowed to refine freely with an isotropic displacement parameter. A reference diffractogram for the crystal structure was generated using Mercury.

### Differential Scanning Calorimetry (DSC)

DSC analysis was carried out on a TA Instruments Discovery DSC instrument equipped with a 50 position auto-sampler. Typically, 0.5-3 mg of each sample, in a pin-holed aluminium pan, was heated at 10° C./min from 25° C. to 300° C. A purge of dry nitrogen at 50 ml/min was maintained over the sample.

The instrument control software was TRIOS, and the data were analysed using TRIOS or Universal Analysis.

### Thermo-Gravimetric Analysis (TGA)

TGA analysis was carried out on a TA Instruments Discovery TGA, equipped with a 25-position auto-sampler. Typically, 5-10 mg of each sample was loaded onto a pre-tared aluminium DSC pan and heated at 10° C./min from ambient temperature to 350° C. A nitrogen purge at 25 ml/min was maintained over the sample.

The instrument control software was TRIOS, and the data were analysed using TRIOS or Universal Analysis.

### Gravimetric Vapour Sorption (GVS)

Sorption isotherms were obtained using a SMS DVS Intrinsic moisture sorption analyser, controlled by DVS Intrinsic Control software. The sample temperature was maintained at 25° C. by the instrument controls. The humidity was controlled by mixing streams of dry and wet nitrogen, with a total flow rate of 200 ml/min. The relative humidity was measured by a calibrated Rotronic probe (dynamic range of 1.0-100% RH), located near the sample. The weight change, (mass relaxation) of the sample as a function of % RH was constantly monitored by a microbalance (accuracy ±0.005 mg).

Typically, 5-30 mg of sample was placed in a tared mesh stainless steel basket under ambient conditions. The sample was loaded and unloaded at 40% RH and 25° C. (typical room conditions). A moisture sorption isotherm was performed as outlined below, in Table 2 (2 scans per complete cycle). The standard isotherm was performed at 25° C. at 10% RH intervals over a 0-90% RH range. Typically, a double cycle (4 scans) was carried out. Data analysis was carried out within Microsoft Excel using the DVS Analysis Suite.

TABLE 2
Moisture Sorption Isotherm Parameters

| Parameter | Value |
|---|---|
| Adsorption - Scan 1 | 40-90 |
| Desorption, Adsorption - Scan 2 | 90-0, 0-40 |
| Intervals (% RH) | 10 |
| Number of Scans | 4 |
| Flow rate (ml/min) | 200 |
| Temperature (° C.) | 25 |
| Stability (° C./min) | 0.2 |
| Sorption Time (hours) | 6 hour time out |
| Number of cycles | 2 |

### Example 2. Preparation of Crystalline Compound 1 Mono-HCl Salt

### Preparation of Compound 1 HCl Salt

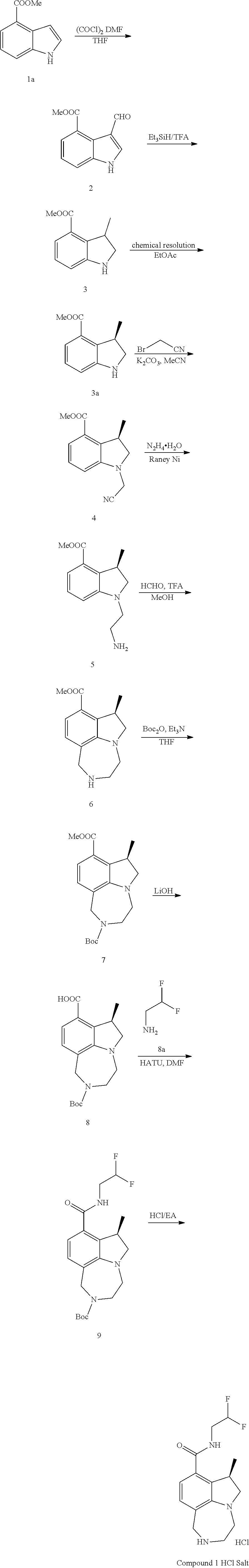

Compound 2: To a mixture of oxalyl dichloride (609 g, 4.80 mol, 420 mL, 1.2 eq) in THE (7.00 L), DMF (418 g, 4.80 mol, 445.00 mL, 1.2 eq) was added drop-wise under N₂ at 0° C., and the mixture was stirred for 0.5 h at 0° C. Then compound 1a (700 g, 4.00 mol, 1.0 eq) which was dissolved in THE (700 mL) was added to the mixture and the mixture was stirred at 25° C. for 1 h. The reaction mixture was adjusted to pH˜8 with NaHCO₃ (aq.) and extracted with EA (10 L*2). The combined organic layer was washed with brine (10 L), dried with Na₂SO₄, filtered and concentrated, the compound 2 (3.18 kg, 15.6 mol, 97.8% yield) (4 parallel reactions) was obtained as a brown solid which was confirmed by NMR. ¹HNMR (400 MHz, DMSO): δ 8.35 (s, 1H), 7.76-7.74 (d, J=8 Hz, 1H), 7.59-7.57 (d, J=8 Hz, 1H), 7.35-7.31 (t, J=8 Hz, 1H), 3.87 (s, 3H).

Compound 3: To a mixture of Et₃SiH (2.41 kg, 20.7 mol, 3.30 L, 6.0 eq) in CF₃COOH (3.00 L) was added compound 2 (700 g, 3.45 mol, 1.0 eq) which was dissolved in TFA (3.00 L) at 30° C., and the mixture was stirred at 30° C. for 3 h. The mixture was concentrated to remove the CF₃COOH, then the residue was poured into EA (5 L), the organic layer was then extracted with water (5 L*3), the combined aqueous phase was adjusted to pH˜8 with Na₂CO₃ (aq.) and extracted with EA (5 L*3). The combined organic phase was washed with brine and dried with anhydrous Na₂SO₄, filtered and concentrated. The compound 3 (1.62 kg, 7.83 mol, 75.6% yield, 92.4% purity) (3 parallel reactions) was obtained as a yellow oil which was confirmed by LCMS.

Compound 3a: A mixture of compound 3 (650 g, 3.40 mol, 1.0 eq) and (−)-Di-p-toluoyl-D-tartaric acid (657 g, 1.70 mol, 0.50 eq) in EA (6.50 L) was stirred at 78° C. for 1 h. Then the mixture was cooled to 25° C. and stirred for 1 h. The reaction mixture was filtered, and the filter-cake was poured into water, then the mixture was adjusted to pH˜8 with Na₂CO₃ (aq.). The mixture was extracted with EA (5 L*2), the combined organic layer was washed with brine (5 L), dried with Na₂SO₄, filtered and concentrated. The compound 3a (700 g, 3.66 mol, 71.8% yield) (3 parallel reactions) was obtained as a yellow oil which was confirmed by SFC.

Compound 4: A mixture of compound 3a (300 g, 1.57 mol, 1.0 eq), 2-bromoacetonitrile (376 g, 3.14 mol, 209 mL, 2.0 eq) and K₂CO₃ (434 g, 3.14 mol, 2.0 eq) in MeCN (2.00 L) was stirred at 55° C. for 6 h. The mixture was filtrated and the filtered was concentrated. The black residue was purified by column chromatography (SiO₂, PE/EA=20:1-10:1) to give compound 4 (710 g, 3.08 mol, 98.2% yield) (2 parallel reactions) as a yellow solid which was confirmed by NMR. ¹HNMR (400 MHz, CDCl₃): δ 7.51-7.49 (d, J=8 Hz, 1H), 7.28-7.25 (t, J=4 Hz, 1H), 6.76-6.74 (d, J=8 Hz, 1H), 4.25-4.21 (br, 1H), 4.05-4.00 (br, 1H), 3.92 (s, 3H), 3.80 (s, 1H), 3.54-3.50 (t, J=8 Hz, 1H), 3.27-3.24 (m, 1H), 1.31-1.29 (d, J=8 Hz, 3H).

Compound 5: To the mixture of compound 4 (100 g, 434 mmol, 1.0 eq) and RANEY NICKEL (100 g, 1.17 mol, 2.7 eq) in MeOH (1.50 L), N₂H₄·H₂ (217 g, 4.34 mol, 211 mL, 10 eq) was added dropwise at 40° C. and the mixture was stirred at 50° C. for 12 h. The mixture was concentrated to remove MeOH, then the mixture was poured into brine (2 L), extracted with EA (2 L*2), and dried with Na₂SO₄ and concentrated to give the compound 5 (608 g, purity: 64% by LCMS) (7 parallel reactions) which was used for the next step without further purification.

Compound 6: A mixture of compound 5 (176 g, 751 mmol, 1.0 eq), HCHO (183 g, 2.25 mol, 168 mL, 37% purity, 3.00 eq) and CF₃COOH (257 g, 2.25 mol, 167 mL, 3.0 eq) in MeOH (800 mL) was stirred at 50° C. for 4 h. The mixture was concentrated to remove the MeOH, then adjusted to pH˜8 with NaHCO₃ (aq.) and extracted with EA (2 L*2), washed with brine (2 L), dried with Na₂SO₄, and concentrated to give the compound 6 (600 g, purity: 69% by LCMS (3 parallel reactions) which was used for the next step without further purification.

Compound 7: To the mixture of compound 6 (300 g, 1.22 mol, 1.0 eq) in THE (1.50 L), 2,2-dimethylpropanoyl 2,2-dimethylpropanoate (340 g, 1.83 mol, 370 mL, 1.50 eq) and Et₃N (308 g, 3.05 mol, 422 mL, 2.5 eq) was added and the mixture was stirred at 25° C. for 16 h. The reaction mixture was concentrated to remove THF. The mixture was poured into water (2 L) and extracted with EA (2 L*3). The combined organic layer was washed with brine (2 L), dried with Na₂SO₄, filtered and concentrated. The yellow residue was purified by column chromatography (SiO₂, PE/EA=100:1-50:1-10:1) to give the compound 7 (274 g, 728 mmol, 29.8% yield, 92% purity) (2 parallel reactions) as a yellow oil which was confirmed by LCMS and NMR. ¹HNMR (400 MHz, CDCl₃): δ 7.38-7.36 (d, J=8 Hz, 1H), 7.07-6.94 (m, 1H), 4.88-4.65 (m, 1H), 4.08-3.99 (m, 2H), 3.88 (s, 3H), 3.43-3.26 (m, 3H), 3.15-3.12 (d, J=12 Hz, 1H), 2.95-2.82 (m, 1H), 1.66-1.65 (d, J=4 Hz, 1H), 1.43-1.39 (d, J=16 Hz, 9H), 1.22-1.20 (d, J=8 Hz, 3H).

Compound 8: To the mixture of compound 7 (160 g, 462 mmol, 1.0 eq) in MeOH (400 mL) and THE (400 mL), LiOH·H₂O (58 g, 1.4 mol, 3.0 eq) which was dissolved in H₂O (200 mL) was added, the mixture was stirred at 25° C. for 10 h. LCMS showed the compound 7 was consumed and the desired product was detected. The mixture was concentrated to remove THE and MeOH, then the mixture poured into water (500 mL), adjusted to pH˜3 with 2 M HCl, filtered and concentrated the filter cake to give the compound 8 (282 g, 848 mmol, 91.8% yield) as a yellow solid.

Compound 9: To the mixture of compound 8 (141 g, 424 mmol, 1.0 eq) in DMF (1.00 L), HATU (193 g, 509 mmol, 1.2 eq), DIEA (164 g, 1.27 mol, 222 mL, 3.0 eq) and compound 8a (41 g, 509 mmol, 1.2 eq) was added, the mixture was stirred at 25° C. for 2 h. The mixture was poured into water (5 L), extracted with EA (1.5 L*2). The combined organic layer was washed with brine (2 L), dried with Na₂SO₄, filtered and concentrated. The yellow residue was purified by column chromatography (PE/EA=100:1-50:1-10:1) to give the compound 9 (250 g, 632 mmol, 74.5% yield) as a yellow oil which was confirmed by NMR. ¹HNMR (400 MHz, CDCl₃): δ 6.99-6.84 (m, 2H), 5.95-5.67 (m, 1H), 3.91-2.93 (m, 11H), 1.40-1.37 (d, J=12 Hz, 9H), 1.19-1.17 (d, J=8 Hz, 3H).

Compound 1 HCl Salt: Compound 9 (250 g, 632 mmol, 1.0 eq) was added to the HCl/EtOAc (1.50 L), and the mixture was stirred at 25° C. for 1 h. The mixture was filtered, and the filter cake was concentrated to give Compound 1 HCl salt (175 g, 527 mmol, 83.4% yield, HCl) as a brown solid which was confirmed by NMR. ¹HNMR (400 MHz, MeOD): δ 7.27-7.25 (d, J=8 Hz, 1H), 7.20-7.18 (d, J=8 Hz, 1H), 6.16-5.88 (m, 1H), 4.52-4.48 (d, J=16 Hz, 1H), 4.36-4.32 (d, J=16 Hz, 1H), 3.88-3.81 (m, 9H), 1.24-1.22 (d, J=8 Hz, 3H).

### Crystalline Compound 1 Mono-HCl Salt

Compound 1 HCl salt (30 mg) was dissolved in methanol (10 vol., 300 μL). The solvent was removed by controlled evaporation in a Genevac HT-6 using the low BP method (20-5 mbar) to give crystalline Compound 1 mono-HCl salt, which was confirmed by XRPD.

### Example 3. Characterization of Crystalline Compound 1 Mono-HCl Salt from Batch 1

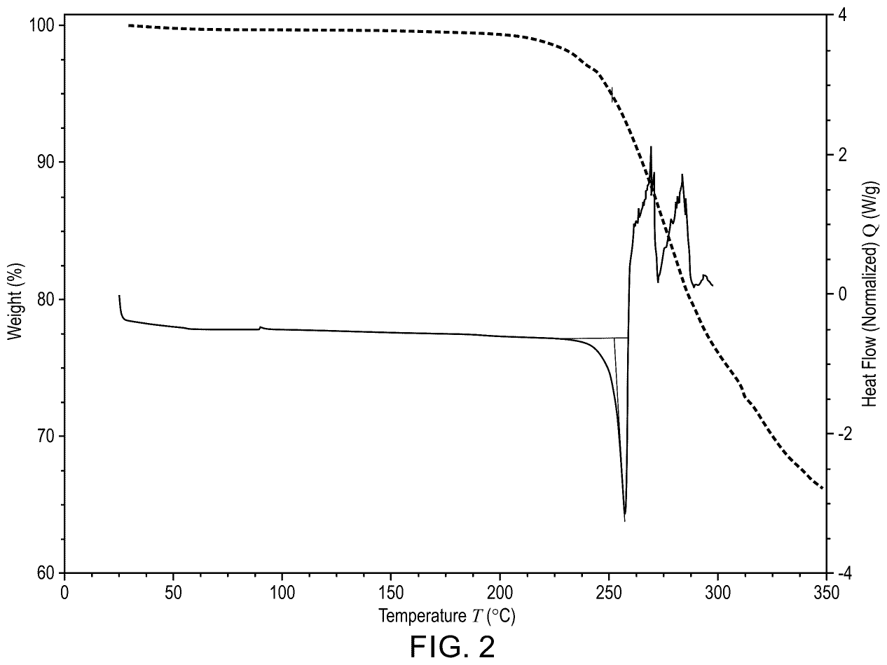

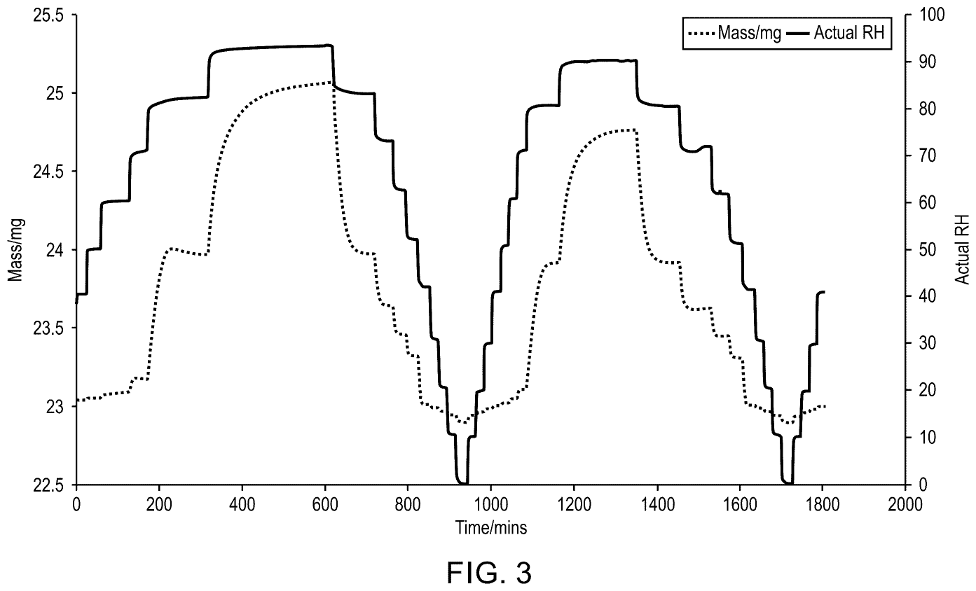

A sample containing crystalline Compound 1 mono-HCl salt from a first batch (“batch 1”) was analyzed by XRPD, TGA, DSC, and GVS. Results are shown in FIG. 1 (XRPD pattern), FIG. 2 (DSC thermogram (solid line) and TGA thermogram (dashed line)), and FIG. 3 (GVS kinetic plot), and are summarized in Table 3, below. The methods used to measure the physical properties of Compound 1 mono-HCl salt from batch 1 are described in Example 1.

TABLE 3
Physical Properties of Crystalline
Compound 1 mono-HCl Salt batch 1

| | |
|---|---|
| XRPD | Main peaks, in terms of 2-theta, at 10.0° ± 0.2°, |
| | 14.5° ± 0.2°, and 22.9° ± 0.2° |
| TGA | Degradation above 251° C. |
| DSC | Endotherm onset 252.1° C. and a maximum at about 257° C. |
| GVS | ~9.4 wt % reversible uptake 0-90% RH |
| | ~8 wt % uptake 70-90% RH |
| | Hysteresis between 40 and 80% RH |

Certain X-ray powder diffraction peaks for crystalline Compound 1 mono-HCl salt are shown in Table 4, below.

TABLE 4
XRPD Peaks for Crystalline Compound 1 mono-HCl Salt

| Pos. [°2-theta] | Rel. Int. [%] |
|---|---|
| 10.0 | 100.0 |
| 13.7 | 4.9 |
| 14.5 | 56.0 |
| 15.5 | 15.9 |
| 16.0 | 12.6 |
| 18.6 | 4.0 |
| 18.9 | 19.2 |
| 19.9 | 25.3 |
| 20.6 | 5.2 |
| 21.1 | 8.4 |
| 21.8 | 19.4 |
| 22.1 | 19.4 |
| 22.4 | 12.3 |
| 22.9 | 48.1 |
| 24.3 | 26.7 |
| 24.5 | 5.2 |
| 25.2 | 16.5 |
| 25.5 | 14.1 |
| 25.8 | 16.5 |
| 27.2 | 11.0 |
| 27.5 | 13.9 |
| 28.2 | 3.6 |
| 28.5 | 5.1 |
| 28.8 | 7.1 |
| 29.3 | 3.9 |
| — | — |

A single crystal of Compound 1 mono-HCl salt having approximate dimensions of 0.30×0.18×0.09 mm was analyzed by single crystal X-ray diffraction. Data collection details are shown in Table 5 below.

TABLE 5
SCXRD Data Collection Details

| | |
|---|---|
| Data collection method | omega scans |
| Theta range for data collection | 3.608 to 70.187° |
| Index ranges | −6 ≤ h ≤ 4, −15 ≤ k ≤ 15, −15 ≤ l ≤15 |
| Reflections collected | 15423 |
| Independent reflections | 3058 [R(int) = 0.0423] |
| Coverage of independent reflections | 100.0% |
| Absorption correction | Semi-empirical from equivalents |
| Max. and min. transmission | 1.00000 and 0.86854 |
| Structure solution technique | Direct methods |
| Structure solution program | SHELXTL (Sheldrick, 2014) |
| Refinement technique | Full-matrix least-squares on F² |
| Refinement program | SHELXL-2014 (Sheldrick, 2014) |
| Function minimized | Σw(Fₒ² − F_{c}²)² |
| Data/restraints/parameters | 3058/109/240 |
| Goodness-of-fit on F² | 1.053 |
| Δ/σₘₐₓ | 0.000 |
| Final R indices | |
| 2936 data; I > 2σ(I) | R1 = 0.0303, wR2 = 0.0746 |
| all data | R1 = 0.0330, wR2 = 0.0769 |
| Weighting scheme | w = 1/[σ² (Fₒ²) + (0.0383P)² + 0.2352P] |
| | where P = (Fₒ² − 2F_{c}²)²/3 |
| Absolute structure parameter | −0.018(10) |
| Extinction coefficient | n/a |
| Largest diff. peak and hole | 0.176 and −0.148 eÅ⁻³ |
| Temperature | 100 K |

The crystal was monoclinic, space group P2₁, with the final R1[I>2σ(I)]=2.02%. Crystal data are shown in Table 6, below.

TABLE 6
Compound 1 mono-HCl Salt Crystal Data

| Empirical formula | C₁₅H₂₀ClF₂N₃O | |
|---|---|---|
| Formula weight | 331.79 | |
| Crystal size | 0.300 × 0.180 × 0.090 mm | |
| Crystal habit | yellow cut block | |
| Crystal system | Monoclinic | |
| Space group | P2₁ | |
| Unit cell dimensions | a = 5.14731(10) Å α = 90° | |
| | b = 12.7849(3) Å β = 99.7707(19)° | |
| | c = 12.4300(3) Å γ = 90° | |
| Volume | 806.13(3) Å³ | |
| Z | 2 | |
| Density (calculated) | 1.367 | Mg/m³ |
| Absorption coefficient | 2.337 | mm⁻¹ |

| | |
|---|---|
| F(000) | 348 |

Atomic coordinates and equivalent isotropic atomic displacement parameters (Å²) of the unit cell are shown in Table 7, below.

TABLE 7
Compound 1 mono-HCl Salt Unit Cell Details

| | x/a | y/b | z/c | U(eq)* |
|---|---|---|---|---|
| Cl1 | 1.26845(12) | 0.88324(7) | 0.04589(5) | 0.0361(2) |
| O1 | 0.1465(4) | 0.37926(18) | 0.39007(14) | 0.0257(4) |
| N1 | −0.0900(4) | 0.36649(19) | 0.22065(19) | 0.0233(5) |
| N2 | 0.8201(5) | 0.81560(18) | 0.15906(18) | 0.0194(5) |
| N3 | 0.6818(4) | 0.70678(18) | 0.37019(17) | 0.0186(5) |
| F1A | 0.0810(10) | 0.1540(4) | 0.2501(5) | 0.061(2) |
| F2A | −0.3223(14) | 0.1011(4) | 0.2322(8) | 0.067(2) |
| C1A | −0.165(2) | 0.1796(9) | 0.2059(11) | 0.041(2) |
| F1B | −0.1036(11) | 0.1456(4) | 0.1549(5) | 0.0567(19) |
| F2B | −0.2921(14) | 0.1091(5) | 0.2960(6) | 0.0537(17) |
| C1B | −0.135(2) | 0.1813(8) | 0.2559(9) | 0.038(2) |
| C2 | −0.2559(6) | 0.2832(2) | 0.2503(2) | 0.0276(6) |
| C3 | 0.1042(5) | 0.4086(2) | 0.2946(2) | 0.0196(5) |
| C4 | 0.2705(5) | 0.4911(2) | 0.2532(2) | 0.0178(5) |
| C5 | 0.2943(5) | 0.4956(2) | 0.1427(2) | 0.0199(5) |
| C6 | 0.4614(5) | 0.5675(2) | 0.1068(2) | 0.0214(5) |
| C7 | 0.6075(5) | 0.6394(2) | 0.1773(2) | 0.0184(5) |
| C8 | 0.5673(5) | 0.6393(2) | 0.2859(2) | 0.0169(5) |
| C9 | 0.4105(5) | 0.5628(2) | 0.3248(2) | 0.0172(5) |
| C10 | 0.8177(5) | 0.7010(2) | 0.1359(2) | 0.0216(6) |
| C11 | 0.9088(5) | 0.8441(2) | 0.2757(2) | 0.0224(6) |
| C12 | 0.7097(5) | 0.8178(2) | 0.3479(2) | 0.0204(5) |
| C13 | 0.5345(5) | 0.6885(2) | 0.4616(2) | 0.0207(5) |
| C14 | 0.4435(5) | 0.5744(2) | 0.4478(2) | 0.0183(5) |
| C15 | 0.6560(5) | 0.5000(2) | 0.5059(2) | 0.0239(6) |

*U(eq) is defined as one third of the trace of the orthogonalized Uᵢⱼ tensor

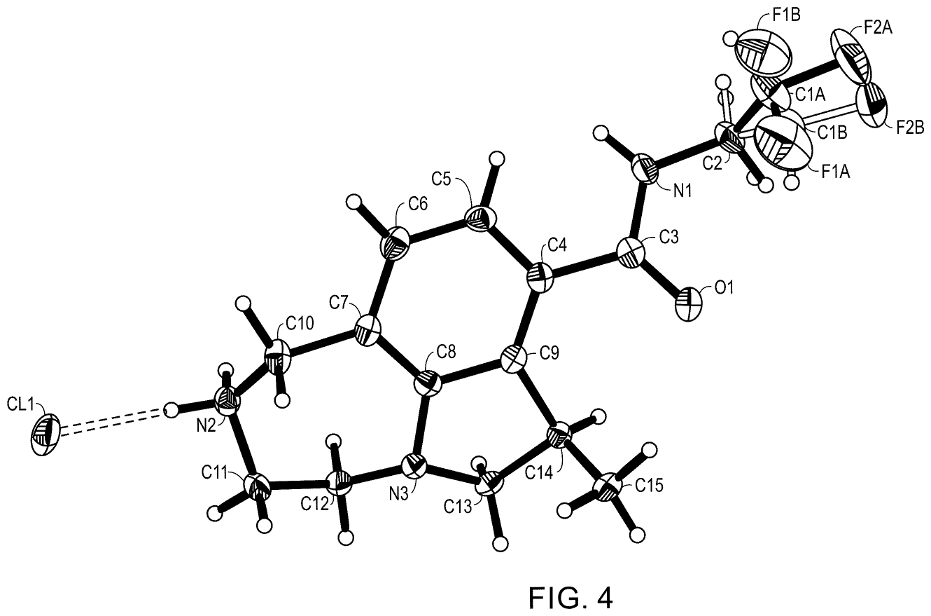

The ball and stick diagram of Compound 1 mono-HCl salt is shown in FIG. 4. The asymmetric unit contains one molecule of protonated compound and one Cl⁻ anion with the 2,2-difluoroethane group disordered and modelled over two sites with 51/49 ratio. The crystal structure was the average of all molecules within the 3D structure as a single unit cell. The modelling of the disorder indicates that both positions of the 2,2-difluoroethane group are equally favored within the crystal structure and that about 50% of molecules have one orientation, and 50% the other.

For the structure shown in FIG. 4, with C14 in the R configuration, the Flack parameter was −0.02(1)³. For the inverted structure with C14 in the S configuration, the Flack parameter=1.01(1).

Determination of the absolute structure using Bayesian statistics on Bijvoet differences revealed that the probability of the absolute structure as presented being correct is 1.000, while the probabilities of the absolute structure being a racemic twin or false are both 0.000. The Flack equivalent and its uncertainty were calculated to be −0.014(7), based on 1453 Bijvoet pairs with a coverage of 99%.

The phenyl ring (C₃-C₈) part of 1,2,3,4,6,7-hexahydro[1,4]diazepino[6,7,1-hi]indo-2-ium moiety had a calculated root mean square deviation (RMSD) from planarity of 0.029, with C7, −0.045 (0.002) Å showing the greatest deviation from planarity (see FIG. 4).

There were three N—H . . . Cl intermolecular hydrogen bonds in the structure between the Cl₋ anion and the nitrogen atoms, N1 and N2. These interactions resulted in an infinite chain propagating along crystallographic a-axis. With the formation of these intermolecular hydrogen bonds, Etters rules were satisfied and there were no further hydrogen bonding interactions and as such the crystal structure packing was further stabilised by Van der Waals interactions only.

There were no other unusual structural features, and the final Fourier difference map was featureless, showing maximal and minimal electron densities of 0.18 and −0.15 eÅ⁻³.

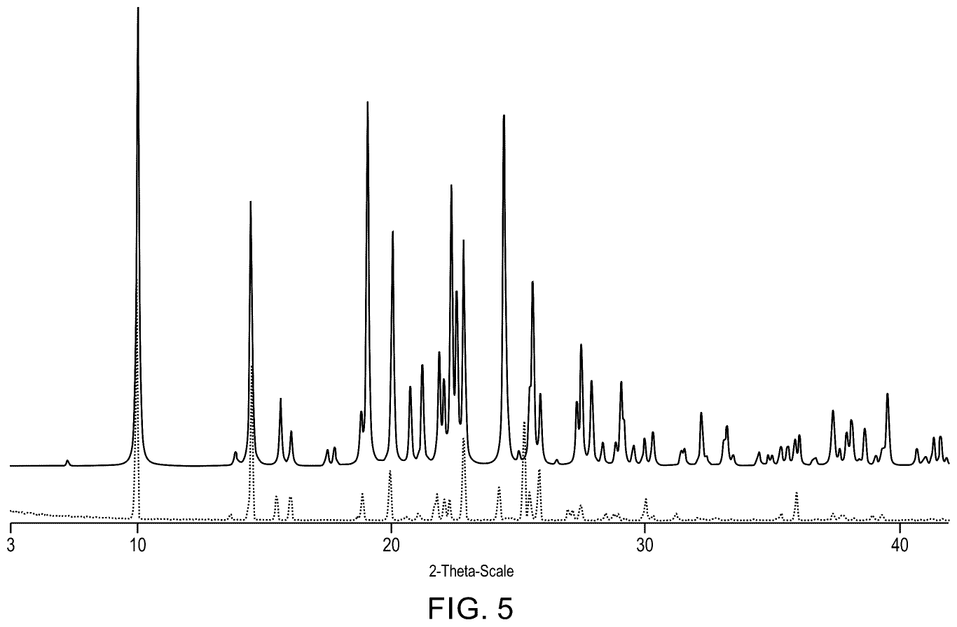

FIG. 5 shows an overlay of the simulated XRPD pattern for Compound 1 mono-HCl salt at 100 K (black line) with the experimental diffractogram at RT (red line), indicating that the single crystal is consistent with the bulk sample. Slight differences in the simulated and experimental diffractograms were attributable to lattice variations with temperature and preferred orientation.

### Example 4. Characterization of Crystalline Compound 1 Mono-HCl Salt from Batch 2

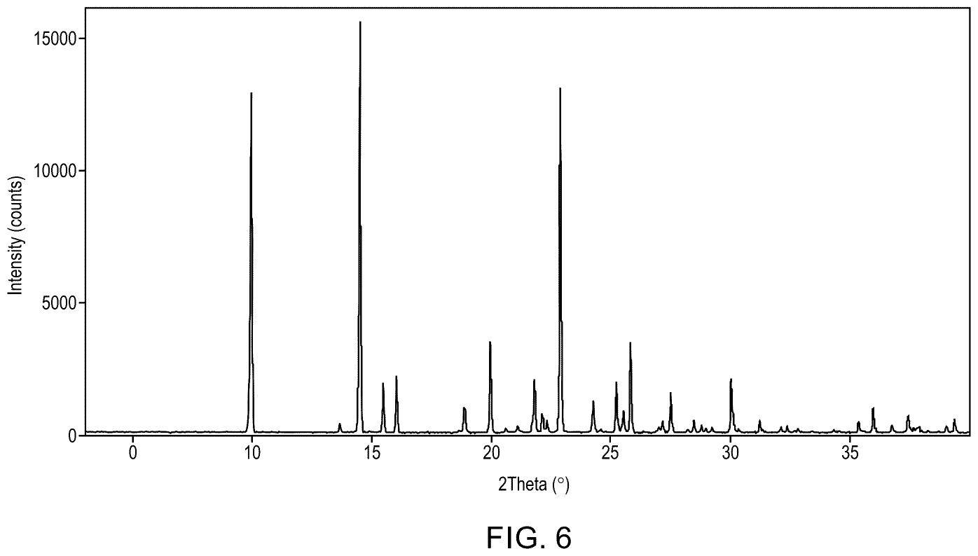

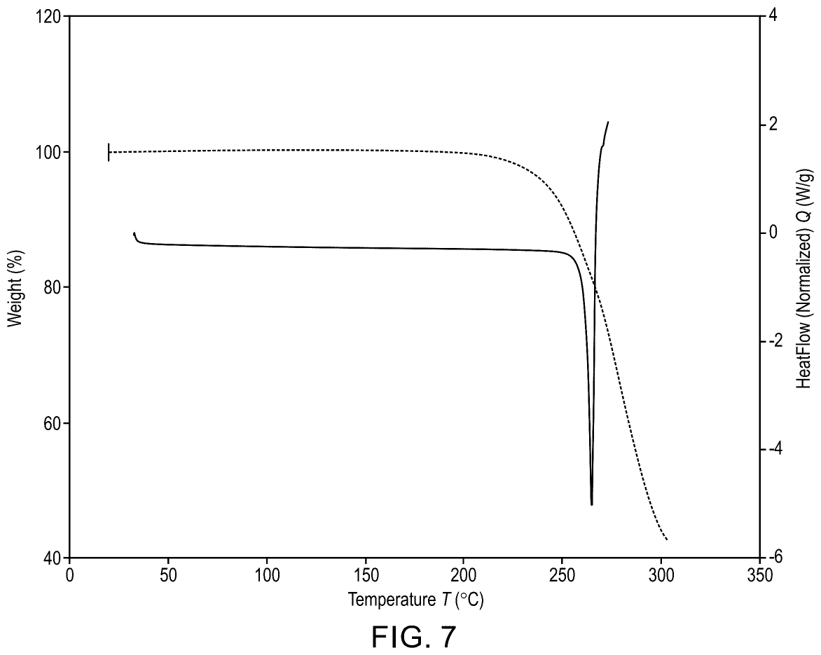

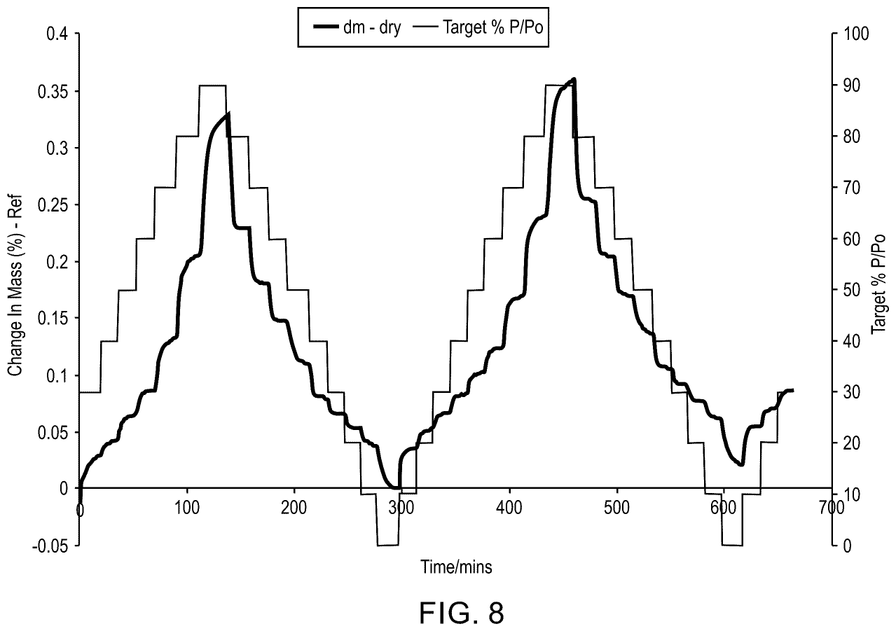

A second batch of crystalline Compound 1 mono-HCl salt (“batch 2”) was analyzed by XRPD, TGA, DSC, and GVS. XRPD results, shown in FIG. 6 and Table 8, confirm that crystalline Compound 1 mono-HCl salt was resynthesized. However, the results shown in FIGS. 7-8 and Table 8 also show variations in hygroscopicity, determined by DVS, and thermal properties, determined by TGA and DSC, between batches. Without wishing to be bound by theory, the differences in results from the two batches is believed to be attributable to impurities in batch 1.

TABLE 8
Physical Properties of batch 2 of Crystalline
Compound 1 mono-HCl Salt

| | |
|---|---|
| XRPD | Main peaks, in terms of 2-theta, at 10.0° ± 0.2°, |
| | 14.5° ± 0.2°, and 22.9° ± 0.2° |
| TGA | Degradation above 200° C. |
| DSC | Endotherm onset 261.7° C. |
| GVS | ~0.2 wt % reversible uptake 0-80% RH |
| | 0.3-0.4 wt % reversible uptake 0-90% RH |
| | No significant hysteresis observed |

Comparison of Table 3 and Table 8 shows that the reversible absorption of 9.4% at 0-90% RH for batch 1 is not a general property of the crystalline form, and instead may be attributable to, for example, impurities in the first batch. This is consistent with characterization of a third batch (“batch 3”), which showed a reversible absorption of 0.25 at 80% RH (GVS kinetic plot not shown). These results suggest that crystalline Compound 1 mono-HCl salt is a slightly to non-hygroscopic compound.

### Example 5. Instruments and Data Collection Parameters for Batch 2 and Batch 3

Batch 2 and batch 3 were characterized by XRPD, TGA, DSC and DVS. Below is listed the instruments and method used for the characterization of the second and third batches of crystalline Compound 1 mono-HCl salt.

### XRPD:

Empyrian diffractometer from Malvern Panalytical using Cu Kα radiation. The sample was gently pressed flat on a zero background plate and measured in reflection mode in the range 3-40° 2⊖.

### DSC:

TA Instruments Discovery DSC. ˜2.5 mg sample was heated 10°/min to 275° C. in a pin-holed aluminum pan under nitrogen flow.

### TGA:

TA Instruments Discovery TGA. ˜3 mg sample was heated 10°/min to 300° C. in an aluminum pan under nitrogen flow.

### DVS

DVS Adventure-1 from SMS. ˜5 mg was measured using 2 cycles between 0% RH and 90% RH in steps of 10% RH.

Although the disclosure has been described with reference to the above examples, it will be understood that modifications and variations are encompassed within the spirit and scope of the disclosure. The various embodiments described above can be combined to provide further embodiments. All the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications, and publications to provide yet further embodiments.

1. The mono-HCl salt of (R)—N-(2,2-difluoroethyl)-7-methyl-1,2,3,4,6,7-hexahydro-[1,4]diazepino[6,7,1-hi]indole-8-carboxamide (Compound 1) which is substantially crystalline.

2. The crystalline salt of claim 1, having an XRPD pattern comprising a peak, in terms of 2-theta, at 10.0°±0.2°.

3. The crystalline salt of claim 1, having an XRPD pattern comprising peaks, in terms of 2-theta, at 10.0°±0.2°, 14.5°±0.2°, and 22.9°±0.2°.

4. The crystalline salt of claim 1, having an XRPD pattern further comprising peaks, in terms of 2-theta, at 19.9°±0.2° and 24.3°±0.2°.

5. The crystalline salt of claim 1, having an XRPD pattern with peaks substantially as shown in FIG. 1.

6. A pharmaceutical composition comprising the crystalline salt of claim 1, and a pharmaceutically acceptable carrier.

7. A method of treating or preventing a 5-hydroxytryptamine (HT)_{2C} receptor-associated disorder in a patient in need thereof, wherein the method comprises administering to the patient the crystalline salt of claim 1.

8. A method of treating or preventing epilepsy in a patient in need thereof, wherein the method comprises administering to the patient the crystalline salt of claim 1.

9. A method of reducing severity of an epileptic seizure in a patient in need thereof, wherein the method comprises administering to the patient the crystalline salt of claim 1.

10. A method of reducing the frequency of epileptic seizures in a patient in need thereof, wherein the method comprises administering to the patient the crystalline salt of claim 1.

11. A method of treating or preventing a seizure disorder in a patient in need thereof, wherein the method comprises administering to the patient the crystalline salt of claim 1.

12. The method of claim 11, wherein the seizure disorder is selected from epilepsy, epilepsy with generalized tonic-clonic seizures, epilepsy with myoclonic absences, frontal lobe epilepsy, temporal lobe epilepsy, Landau-Kleffner Syndrome, Rasmussen's syndrome, Dravet syndrome, Doose syndrome (epilepsy with myoclonic atonic seizures (EM AS)), CDKL5 deficiency disorder (CDKL5 encephalopathy, or CDD), infantile spasms (West syndrome), juvenile myoclonic epilepsy (JME), vaccine-related encephalopathy, intractable childhood epilepsy (ICE), Lennox-Gastaut syndrome (LGS), Rett syndrome, Ohtahara syndrome (early infantile DEE, EIDEE), childhood absence epilepsy, essential tremor, acute repetitive seizures, benign rolandic epilepsy, status epilepticus, refractory status epilepticus, super-refractory status epilepticus (SRSE), PCDH19 pediatric epilepsy, drug withdrawal induced seizures, alcohol withdrawal induced seizures, increased seizure activity and breakthrough seizures.

13. A method of treating developmental and epileptic encephalopathy (DEE) in a patient in need thereof, wherein the method comprises administering to the patient the crystalline salt of claim 1.

14. (canceled)

15. The method of claim 13, wherein the DEE is selected from Ohtahara syndrome (EIDEE), Lennox-Gastaut syndrome, Dravet syndrome, Doose syndrome (EM AS), West syndrome (infantile spasms), Landau-Kleffner syndrome, tuberous sclerosis complex, CDKL5 encephalopathy (CDKL5 deficiency disorder), dup15q syndrome, SCN2A related epilepsies, SCN8A related epilepsies, KCNQ2 related epilepsies, KCNQ3 related epilepsies, Angelman syndrome, KCNT1 related epilepsies, SynGAP1 related epilepsies, Rett syndrome, PCDH19 epilepsy, ring 14 syndrome, ring 20 syndrome, CHD2 encephalopathy, early myoclonic encephalopathy, epilepsy of infancy with migrating focal seizures, and epileptic encephalopathy with continuous spike-wave.

16. A method of treating a refractory epilepsy in a patient in need thereof, wherein the method comprises administering to the patient the crystalline salt of claim 1.

17. A process for preparing the crystalline salt of claim 1, comprising precipitating the mono-HCl salt of Compound 1 from a solvent.

18. The process of claim 17, wherein the solvent comprises an alcohol.

19. (canceled)

20. The process of claim 17, wherein the solvent comprises acetonitrile.

21. The process of claim 17, wherein the precipitating comprises evaporating a solution comprising Compound 1 and hydrochloric acid.

22. The mono-HCl salt of (R)—N-(2,2-difluoroethyl)-7-methyl-1,2,3,4,6,7-hexahydro-[1,4]diazepino[6,7,1-hi]indole-8-carboxamide (Compound 1), prepared by the process of claim 17.
